(19) United States
(12) Patent Application Publication
(10) Pub. No.: US 2024/0092800 A1
(43) Pub. Date: Mar. 21, 2024

(54) USP13 INHIBITORS AND METHODS OF USE THEREOF

(71) Applicant: GEORGETOWN UNIVERSITY, Washington, DC (US)

(72) Inventors: Charbel MOUSSA, Washington, DC (US); Christian WOLF, Washington, DC (US); Balaraman KALUVU, Washington, DC (US)

(73) Assignee: GEORGETOWN UNIVERSITY, Washington, DC (US)

(21) Appl. No.: 18/260,721

(22) PCT Filed: Jan. 14, 2022
(86) PCT No.: PCT/US2022/012447

§ 371 Date: Jul. 7, 2023

Related U.S. Application Data
(60) Provisional application No. 63/137,425, filed on Jan. 14, 2021.

(51) Int. Cl. C07D 495/04; A61K 45/06; A61P 25/28; C07D 215/233; C07D 215/44; C07D 311/68

(52) U.S. Cl. CPC C07D 495/04; A61K 45/06; A61P 25/28; C07D 215/233; C07D 215/44; C07D 311/68

(57) ABSTRACT

Novel ubiquitin specific protease 13 (USP13) inhibitors are provided, along with methods for their use. The USP13 inhibitors described herein are useful in treating and/or preventing USP13-related diseases, such as neurodegenerative diseases and cancer. Also provided are methods for inhibiting USP13 in a cell using the compounds and compositions described herein.

### PRIOR RELATED APPLICATION

This application claims the benefit of U.S. Provisional Application No. 63/137,425 filed on Jan. 14, 2021, which is hereby incorporated by reference in its entirety.

### BACKGROUND

Ubiquitin linkage is involved in most cellular processes and signaling pathways. Ubiquitin specific protease (USP)-13 (USP13) is a de-ubiquitinase member of the cysteine-dependent protease superfamily. Specifically, USP13 is a ubiquitin-specific enzyme that cleaves ubiquitin off protein substrates to reverse ubiquitin-mediated protein degradation. Ubiquitin targets proteins to major degradation pathways, including the proteasome and the lysosome. In melanoma cells, USP13 regulates the degradation of several proteins primarily via ubiquitination and de-ubiquitination. The role of USP13 in regulating the ubiquitination and de-ubiquitination cycle and initiation and control of autophagy and protein degradation are integral to cell homeostasis and survival.

### SUMMARY

Provided herein are novel ubiquitin specific protease 13 (USP13) inhibitors, along with methods for their use. The USP13 inhibitors described herein are useful in treating and/or preventing USP13-related diseases, such as neurodegenerative diseases and cancer. Also provided are methods for inhibiting USP13 in a cell using the compounds and compositions described herein.

Small molecule USP13 inhibitors include compounds of the following formula:

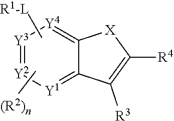

and pharmaceutically acceptable salts or prodrugs thereof In these compounds, n is 0, 1, or 2; L is S, O, or NR⁷; X is S, O, NR⁸, or CR⁵═CR⁶; Y¹, Y², Y³, and Y⁴ are each independently N or CR, wherein R is selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, and wherein at least one of Y¹, Y², Y³, and Y⁴ is N; R¹ is substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl; each R² is independently selected from the group consisting of halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl; R³, R⁴, R⁵, and R⁶ are each independently selected from the group consisting of hydrogen, halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl; and R⁷ and R⁸ are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Optionally, the compound has a formula as shown below:

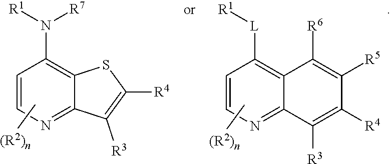

Optionally, the compound is selected from the group consisting of:

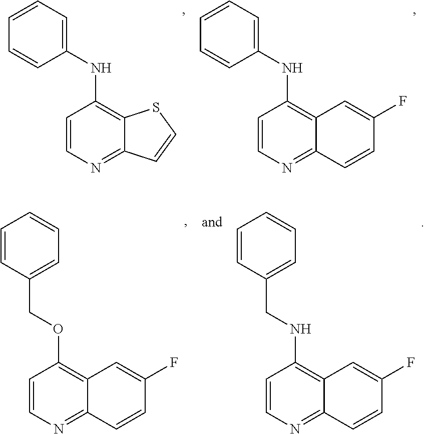 [SMILES: c1ccc(Nc2ccnc3ccsc23)cc1 ; Fc1ccc2nccc(Nc3ccccc3)c2c1 ; Fc1ccc2nccc(OCc3ccccc3)c2c1 ; Fc1ccc2nccc(NCc3ccccc3)c2c1]

Small molecule USP13 inhibitors as described herein also include compounds of the following formula:

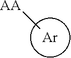

and pharmaceutically acceptable salts and prodrugs thereof, wherein AA is an amino acid or ester thereof; and Ar is substituted or unsubstituted aryl or substituted or unsubstituted heteroaryl, wherein AA is covalently bonded to Ar through an amino group of AA. Optionally, the amino acid or ester thereof is a natural amino acid or ester thereof or an unnatural amino acid or ester thereof.

Optionally, the compound has the following formula:

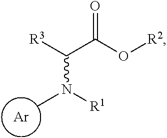

wherein
Ar is substituted or unsubstituted aryl or substituted or unsubstituted heteroaryl; and R¹ and R² are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl; and R³ is hydrogen, halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl.

Optionally, the compound has the following formula:

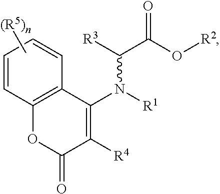

wherein
n is 0, 1, 2, 3, or 4; R⁴ is selected from the group consisting of hydrogen, halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl; and each R⁵ is independently selected from the group consisting of halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Optionally, the compound is selected from the group consisting of:

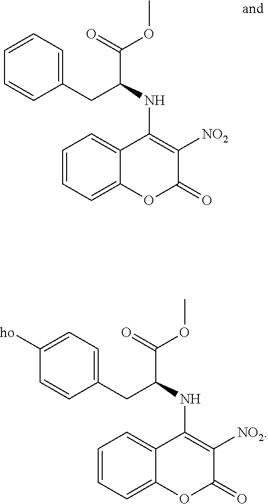

Also described herein is a pharmaceutical composition comprising a compound as described herein and a pharmaceutically acceptable carrier.

Further described herein is a kit comprising a compound or a pharmaceutical composition as described herein.

Methods of treating or preventing a USP13-related disease in a subject are also provided herein. A method of treating or preventing a USP13-related disease in a subject comprises administering to the subject an effective amount of a compound or a pharmaceutical composition as described herein. Optionally, the method further comprises selecting a subject having a USP13-related disease.

Optionally, the USP13-related disease is a neurodegenerative disease (e.g., amyotrophic lateral sclerosis, Alzheimer's disease (AD), Parkinson's disease (PD), frontotemporal dementia, Huntington's disease, mild cognitive impairment, an α-synucleinopathy, a Tauopathy, or a pathology associated with intracellular accumulation of TDP-43). The methods can further comprise selecting a subject having a neurodegenerative disease. The methods can further comprise administering a second therapeutic agent to the subject (e.g., levadopa, a dopamine agonist, an anticholinergic agent, a monoamine oxidase inhibitor, a COMT inhibitor, amantadine, rivastigmine, an NMDA antagonist, a cholinesterase inhibitor, riluzole, an anti-psychotic agent, an antidepressant, or tetrabenazine).

Optionally, the USP13-related disease is cancer (e.g., pancreatic cancer, breast cancer, brain cancer, lung cancer, prostate cancer, colorectal cancer, thyroid cancer, renal cancer, adrenal cancer, liver cancer, neurofibromatosis 1, lymphoma, or leukemia). The methods can further comprise selecting a subject having cancer. The methods can further comprise administering a second therapeutic agent to the subject (e.g., a chemotherapeutic agent or radiation).

Methods of inhibiting USP13 in a cell are also provided herein. A method of inhibiting USP13 in a cell comprises contacting a cell with an effective amount of a compound as described herein.

Methods of reducing alpha-synuclein levels in a cell are also provided herein. A method of reducing alpha-synuclein levels in a cell comprises contacting a cell with an effective amount of a compound as described herein. Optionally, the contacting can be performed in vitro or in vivo.

The details of one or more embodiments are forth in the drawings and the description below. Other features, objects, and advantages will be apparent from the description and drawings, and from the claims.

### DESCRIPTION OF THE DRAWINGS

FIGS. 1A-1F are graphs showing the data obtained from MTT and LDH assays in human SHSY5Y neuroblastoma cells treated with BK50118-A (FIG. 1A), BK50118-B (FIG. 1B), BK50118-C (FIG. 1C), CL3-514 (FIG. 1D), CL3-512 (FIG. 1E) and CL3-499 (FIG. 1F). N=3-4 per group.

FIGS. 2A-2F are graphs showing the USP13 activity via ELISA in human SHSY5Y neuroblastoma cells treated with BK50118-A (FIG. 2A), BK50118-B (FIG. 2B), BK50118-C (FIG. 2C), CL3-514 (FIG. 2D), CL3-512 (FIG. 2E) and CL3-499 (FIG. 2F). N=3-9 per group.

FIGS. 3A-3F represent the ELISA levels of alpha-synuclein in human SHSY5Y neuroblastoma cells treated with BK50118-A (FIG. 3A), BK50118-B (FIG. 3B), BK50118-C (FIG. 3C), CL3-514 (FIG. 3D), CL3-512 (FIG. 3E) and CL3-499 (FIG. 3F). N=3-9 per group.

FIGS. 4A-N show that BK50118-C significantly reduced alpha-synuclein but not p-tau levels in TgA53T mice. Male and female TgA53T mice were treated with intraperitoneal injection of vehicle or BK50118-C at the daily dosage of 10 mg/Kg or 40 mg/Kg for 7 days. WB of midbrain lysates showing (A) the levels of alpha-synuclein relative to actin on 4-12% SDS-NuPAGE gel in the above mice. (B) The first blot is the WB densitometry. The 2nd and 3rd blots are IP alpha-synuclein (syn) or ubiquitin. ELISA showing (C) the levels of alpha-synuclein and (D) ptau396 following BK50118-C treatment. Asterisks indicate statistic significant difference *p<0.05 or **p<0.01 vs. corresponding vehicle. Ordinary one-way ANOVA was used for the analysis. N=3-4 mice per group. DAB +Nissl staining. C significantly reduced alpha-synuclein levels in cortex (F/H) and striatum (K/M) compared to the corresponding vehicles (E/G) or (J/L), verified by the quantification of alpha-synuclein positive cells in cortex (I) and striatum (N). Two-tailed student t test was used for analysis, and * indicates p<0.05 vs. vehicle. N=3-4 per group. All values presented as Mean±SD. Scale bars: 100 μm (E,F,J,K) or 200 μm (G,H,L,M).

FIGS. 5A-N show that BK50118-C significantly increased alpha-synuclein ubiquitination but had minimal effects on tyrosine hydroxylase (TH) levels in striatum of TgA53T mice. Male and female TgA53T mice were treated with intraperitoneal injection of vehicle or BK50118-C at the daily dosage of 40 mg/Kg for 7 days. Immunochemistry assay of 20 μm thick brain sections showed alpha-synuclein, ubiquitination, and DAPI staining in the striatum of TgA53T mice treated with either DMSO (A,C,E) or BK50118-C (B,D,F). BK50118-C increased alpha-synuclein ubiquitination, verified by optic density of co-localization (G). Immunostaining showed TH and DAPI staining in vehicle DMSO (H,J,L) and BK50118-C (I,K,M). BK50118-C had minimal effects on TH levels in striatum, verified by quantification of optic density (N). Asterisk indicates statistic significant difference * p<0.05; two-tailed student's t test was used for analysis. N=3-4 mice per group. All values presented as Mean±SD. Scale bars: 200 μm.

FIGS. 6A-I show that BK50118-C improved cell survivals in TgA53T mice. Male and female TgA53T mice were treated with intraperitoneal injection of vehicle or BK50118-C at the daily dosage of 40 mg/Kg for 7 days. Nissl staining showed BK50118-C significantly increased the neuron counts in cortex B), striatum E) and substantia nigra (SN) H) compared to corresponding vehicles A), D) and G), verified by quantification of Nissl+ cells in cortex C), striatum F) and SN I). Asterisk indicates statistic significant difference vs vehicle. *p<0.05 vs vehicle. Two-tailed student's t test was used for analysis. N=3-4 mice per group. All values presented as Mean±SD.

### DETAILED DESCRIPTION

Ubiquitin specific protease (USP)-13 is a de-ubiquitinase which cleaves ubiquitin off the substrate or protein. In melanoma cells, USP13 regulates the degradation of several proteins primarily via ubiquitination and de-ubiquitination. The role of USP13 in regulating the ubiquitination and de-ubiquitination cycle and initiation and control of autophagy and protein degradation are integral to cell homeostasis and survival. In addition, USP13 is overexpressed in postmortem Alzheimer's disease (AD) and Parkinson's disease (PD) brains. A balance of ubiquitination and de-ubiquitination is important for toxic protein degradation in neurodegenerative diseases. USP13 knockdown increases alpha-synuclein ubiquitination and clearance and regulates parkin function in PD models. USP13 knockdown increases p-tau ubiquitination and facilitates the clearance of p-tau and Aβ in AD models. USP13 thus plays a critical role in regulating protein clearance in neurodegeneration.

Provided herein are novel ubiquitin specific protease 13 (USP13) inhibitors, along with methods for their use. The USP13 inhibitors described herein are useful in treating and/or preventing USP13-related diseases, such as neurodegenerative diseases and cancer. Also provided are methods for inhibiting USP13 in a cell using the compounds and compositions described herein.

I. Compounds

A class of USP13 inhibitors described herein is represented by Formula I:

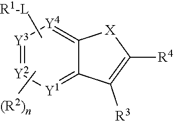

and pharmaceutically acceptable salts or prodrugs thereof.

In Formula I, n is 0, 1, or 2.

Also in Formula I, L is S, O, or NR⁷.

Additionally in Formula I, X is S, O, NR⁸, or CR⁵═CR⁶.

Also in Formula I, Y¹, Y², Y³, and Y⁴ are each independently N or CR, wherein R is selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl. In some examples, at least one of Y¹, Y², Y³, and Y⁴ is N. In some examples, only one of Y¹, Y², Y³, and Y⁴ is N.

Further in Formula I, R¹ is substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl.

Also in Formula I, each R² is independently selected from the group consisting of halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Additionally in Formula I, R³, R⁴, R⁵, and R⁶ are each independently selected from the group consisting of hydrogen, halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Further in Formula I, R⁷ and R⁸ are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

In some cases, the compounds according to Formula I are represented by Structure I-A:

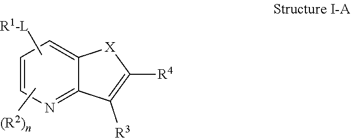

In Structure I-A, n, L, X, R¹, R², R³, R⁴, and R⁷ are as defined above for Formula I.

In some cases, the compounds according to Formula I are represented by Structure I-B:

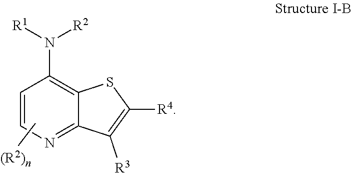

In Structure I-B, n, R¹, R², R³, R⁴, and R⁷ are as defined above for Formula I. Optionally, in Structure I-B, n is 0. Optionally, in Structure I-B, R³, R⁴, and R⁷ are each hydrogen. Optionally, in Structure I-B, R¹ is aryl.

In some cases, the compounds according to Formula I are represented by Structure I-C:

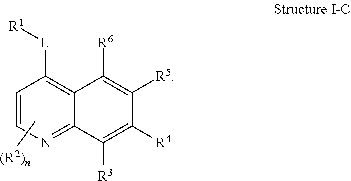

In Structure I-C, n, R¹, R², R³, R⁴, R⁵, R⁶, and L are as defined above for Formula I. Optionally, in Structure I-C, n is 0. Optionally, in Structure I-C, R³, R⁴, and R⁶ are each hydrogen. Optionally, in Structure I-C, R⁵ is halogen (e.g., fluoro, chloro, bromo, or iodo). Optionally, in Structure I-C, L is NH or O. Optionally, in Structure I-C, R¹ is benzyl or phenyl.

Examples of Formula I include the following compounds:

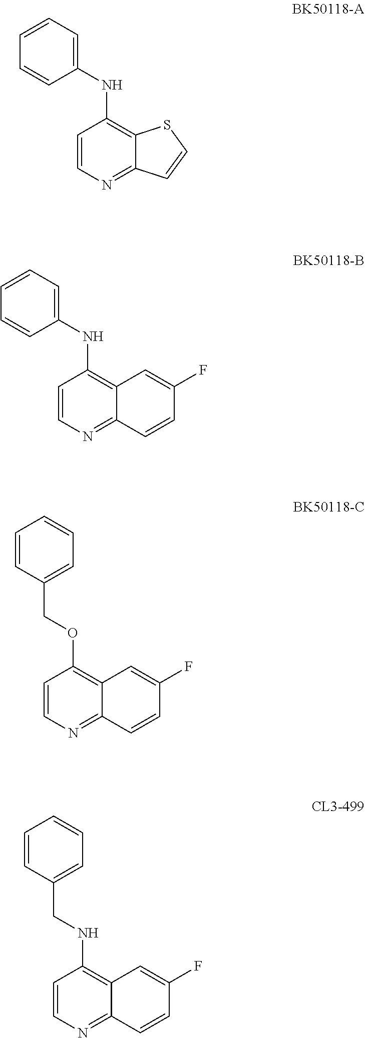 [SMILES: c1ccc(Nc2ccnc3ccsc23)cc1 ; Fc1ccc2nccc(Nc3ccccc3)c2c1 ; Fc1ccc2nccc(OCc3ccccc3)c2c1 ; Fc1ccc2nccc(NCc3ccccc3)c2c1]

A class of USP13 inhibitors described herein is represented by Formula II:

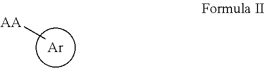

and pharmaceutically acceptable salts or prodrugs thereof.

In Formula II, AA is an amino acid or ester thereof. Optionally, the amino acid or ester thereof is a naturally occurring amino acid, which is also referred to herein as a natural amino acid, or ester thereof. Optionally, the naturally occurring amino acid can be selected from the group consisting of alanine, valine, leucine, isoleucine, proline, tryptophan, phenylalanine, methionine, glycine, serine, tyrosine, threonine, cysteine, asparagine, glutamine, aspartic acid, glutamic acid, lysine, arginine, and histidine.

Optionally, the amino acid or ester thereof is an unnatural amino acid, which is also referred to herein as a non-natural amino acid, or ester thereof. As used herein, the term unnatural amino acid encompasses all amino acid-like compounds that are similar in structure and/or overall shape to one or more of the twenty naturally occurring amino acids. For example, the side chain in the unnatural amino acids can include an alkyl, aryl, aryl halide, vinyl halide, alkyl halide, acetyl, ketone, aziridine, nitrile, nitro, halide, acyl, keto, azido, hydroxyl, hydrazine, cyano, halo, hydrazide, alkenyl, alkynyl, ether, thioether, epoxide, sulfone, boronic acid, boronate ester, borane, phenylboronic acid, thiol, seleno, sulfonyl, borate, boronate, phospho, phosphono, phosphine, heterocyclic, pyridyl, naphthyl, benzophenone, a constrained ring such as a cyclooctyne, thioester, enone, imine, aldehyde, ester, thioacid, hydroxylamine, amino, carboxylic acid, alpha-keto carboxylic acid, alpha or beta unsaturated acids and amides, glyoxyl amide, or organosilane group, or the like. Suitable unnatural amino acids are described in, e.g., Unnatural Amino Acids: Methods and Protocols (Methods in Molecular Biology), Volume 794, Pollegioni and Servi, eds., Springer (2012), which is incorporated herein by reference in its entirety, at least with respect to its description of unnatural amino acids.

The side chains of the amino acid or ester thereof can be in either the (R) or the (S) configuration (or D- or L-configuration).

Also in Formula II, Ar is substituted or unsubstituted aryl or substituted or unsubstituted heteroaryl.

Additionally in Formula II, the AA is covalently bonded to Ar through an amino group of AA.

In some cases, the compounds according to Formula II are represented by Structure II-A:

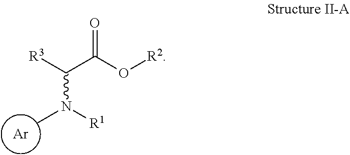

In Structure II-A, Ar is substituted or unsubstituted aryl or substituted or unsubstituted heteroaryl.

Also in Structure II-A, R¹ and R² are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Additionally in Structure II-A, R³ is hydrogen, halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl.

In some cases, the compounds according to Formula II are represented by Structure II-B:

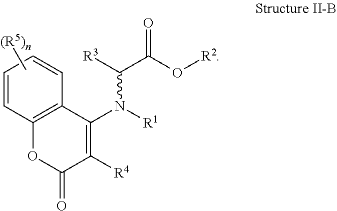

In Structure II-B, R¹, R², and R³ are as defined above for Structure II-A.

Also in Structure II-B, n is 0, 1, 2, 3, or 4.

Additionally in Structure II-B, R⁴ is selected from the group consisting of hydrogen, halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Further in Structure II-B, each R⁵ is independently selected from the group consisting of halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Optionally, in Structure II-B, R¹ is hydrogen. Optionally, in Structure II-B, R² is methyl. Optionally, in Structure II-B, R³ is substituted or unsubstituted benzyl. Optionally, in Structure II-B, R⁴ is nitro. Optionally, in Structure II-B, n is 0.

Examples of Formula II include the following compounds:

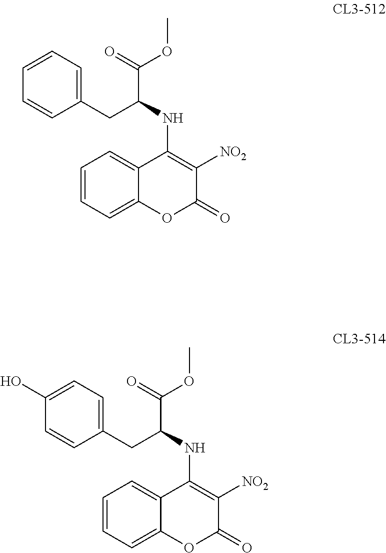 [SMILES: COC(=O)[C@H](Cc1ccccc1)Nc1c([N+](=O)[O-])c(=O)oc2ccccc12 ; COC(=O)[C@H](Cc1ccc(O)cc1)Nc1c([N+](=O)[O-])c(=O)oc2ccccc12]

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bound through a single, terminal ether linkage. The term aryloxy as used herein is an aryl group bound through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryloxy, cycloalkyloxy, and heterocycloalkyloxy group, respectively, bound through a single, terminal ether linkage.

The term hydroxy as used herein is represented by the formula —OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, aryloxy, amino, alkyl, alkenyl, alkynyl, aryl, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

II. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions may vary with the particular reactants or solvents used, but such conditions can be determined by one skilled in the art.

Variations on Formula I and Formula II and the compounds described herein include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of ordinary skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H-NMR or ¹³ C-NMR), infrared spectroscopy (IR), spectrophotometry (e.g., UV-visible), or mass spectrometry (MS), or by chromatography such as high performance liquid chromatography (HPLC) or thin layer chromatography (TLC).

Exemplary methods for synthesizing the compounds as described herein are provided below in Example 1 below.

III. Pharmaceutical Formulations

The compounds described herein or derivatives thereof can be provided in a pharmaceutical composition. Depending on the intended mode of administration, the pharmaceutical composition can be in the form of solid, semi-solid, or liquid dosage forms, such as, for example, tablets, suppositories, pills, capsules, powders, liquids, aerosols, or suspensions, preferably in unit dosage form suitable for single administration of a precise dosage. The compositions will include a therapeutically effective amount of the compound described herein or derivatives thereof in combination with a pharmaceutically acceptable carrier and, in addition, can include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012). Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, New Jersey), polyethylene glycol (PEG), and PLURONICS™ (BASF; Florham Park, NJ).

Compositions containing one or more of the compounds described herein or pharmaceutically acceptable salts or prodrugs thereof suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions can also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like can also be included. Prolonged absorption of the injectable pharmaceutical form can be brought about by the use of agents delaying absorption, for example, aluminum monostearate and gelatin.

Solid dosage forms for oral administration of the compounds described herein or pharmaceutically acceptable salts or prodrugs thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid compositions of a similar type can also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They can contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or pharmaceutically acceptable salts or prodrugs thereof include pharmaceutically acceptable emulsions, solutions, suspensions, syrups, and elixirs. In addition to the active compounds, the liquid dosage forms can contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or pharmaceutically acceptable salts or prodrugs thereof for rectal administrations are optionally suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of the compounds described herein or derivatives thereof include ointments, powders, sprays, inhalants, and skin patches. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the compounds described herein can be contained in a drug depot. A drug depot comprises a physical structure to facilitate implantation and retention in a desired site (e.g., a synovial joint, a disc space, a spinal canal, abdominal area, a tissue of the patient, etc.). The drug depot can provide an optimal concentration gradient of the compound at a distance of up to about 0.1 cm to about 5 cm from the implant site. A depot, as used herein, includes but is not limited to capsules, microspheres, microparticles, microcapsules, microfibers particles, nanospheres, nanoparticles, coating, matrices, wafers, pills, pellets, emulsions, liposomes, micelles, gels, antibody-compound conjugates, protein-compound conjugates, or other pharmaceutical delivery compositions. Suitable materials for the depot include pharmaceutically acceptable biodegradable materials that are preferably FDA approved or GRAS materials. These materials can be polymeric or non-polymeric, as well as synthetic or naturally occurring, or a combination thereof. The depot can optionally include a drug pump.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.0001 to about 200 mg/kg of body weight of active compound per day, which may be administered in a single dose or in the form of individual divided doses, such as from 1 to 4 times per day. Alternatively, the dosage amount can be from about 0.01 to about 150 mg/kg of body weight of active compound per day, about 0.1 to 100 mg/kg of body weight of active compound per day, about 0.5 to about 75 mg/kg of body weight of active compound per day, about 0.5 to about 50 mg/kg of body weight of active compound per day, about 0.01 to about 50 mg/kg of body weight of active compound per day, about 0.05 to about 25 mg/kg of body weight of active compound per day, about 0.1 to about 25 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, about 5 mg/kg of body weight of active compound per day, about 2.5 mg/kg of body weight of active compound per day, about 1.0 mg/kg of body weight of active compound per day, or about 0.5 mg/kg of body weight of active compound per day, or any range derivable therein. Optionally, the dosage amounts are from about 0.01 mg/kg to about 10 mg/kg of body weight of active compound per day. Optionally, the dosage amount is from about 0.01 mg/kg to about 5 mg/kg. Optionally, the dosage amount is from about 0.01 mg/kg to about 2.5 mg/kg.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

IV. Methods of Use

Provided herein are methods to treat or prevent a USP13-related disease in a subject. The methods include administering to a subject an effective amount of one or more of the compounds or compositions described herein, or a pharmaceutically acceptable salt or prodrug thereof. Effective amount, when used to describe an amount of compound in a method, refers to the amount of a compound that achieves the desired pharmacological effect or other biological effect. The effective amount can be, for example, the concentrations of compounds at which USP13 is inhibited in vitro, as provided herein. Also contemplated is a method that includes administering to the subject an amount of one or more compounds described herein such that an in vivo concentration at a target cell in the subject corresponding to the concentration administered in vitro is achieved.

The compounds and compositions described herein or pharmaceutically acceptable salts thereof are useful for treating USP13-related diseases in humans, including, without limitation, pediatric and geriatric populations, and in animals, e.g., veterinary applications.

Optionally, the USP13-related disease is a neurodegenerative disease, such as a neurodegenerative disease of the central nervous system. Such diseases include, but are not limited to, amyotrophic lateral sclerosis, Alzheimer's disease (AD), Parkinson's disease (PD), frontotemporal dementia, Huntington's disease, mild cognitive impairment, an α-synucleinopathy, a Tauopathy, or a pathology associated with intracellular accumulation of TDP-43.

Optionally, the USP13-related disease is cancer. As used herein, cancer refers to any cellular disorder in which the cells proliferate more rapidly than normal tissue growth. A proliferative disorder includes, but is not limited to, neoplasms, which are also referred to as tumors. A neoplasm can include, but is not limited to, pancreatic cancer, breast cancer, brain cancer (e.g., glioblastoma), lung cancer, prostate cancer, colorectal cancer, thyroid cancer, renal cancer, adrenal cancer, liver cancer, neurofibromatosis 1, and leukemia. A neoplasm can be a solid neoplasm (e.g., sarcoma or carcinoma) or a cancerous growth affecting the hematopoietic system (e.g., lymphoma or leukemia). Other proliferative disorders include, but are not limited to neurofibromatosis.

Optionally, the USP13-related disease is a myodegenerative disease, a prion disease, or a transmissible spongiform encephalopathy. In the methods provided herein, myodegenerative diseases include, but are not limited to, inclusion body myositis (IBM), spinal-bulbar muscular atrophy (SBMA), and motor neuron disease (MND). In the methods provided herein, prion diseases or transmissible spongiform encephalopathies (TSEs) include, but are not limited to, Creutzfeldt-Jakob disease (CJD), Variant Creutzfeldt-Jakob disease (vCJD), Gerstmann-Straussler-Scheinker syndrome, fatal familial insomnia and kuru in humans. Animal prion diseases include, but are not limited to, scrapie, bovine spongiform encephalopathy (BSE), chronic wasting disease (CWD), transmissible mink encephalopathy, feline spongiform encephalopathy and ungulate spongiform encephalopathy.

One of skill in the art would know how to select a subject with a disorder associated with USP13. For example, and not to be limiting, one of skill in the art knows how to diagnose a subject with or at risk of developing a neurodegenerative disease. For example, one or more of the following tests can be used, including genetic tests (e.g., identification of a mutation in TDP-43 gene) or familial analysis (e.g., family history), central nervous system imaging (e.g., magnetic resonance imaging and positron emission tomography), clinical or behavioral tests (e.g., assessments of muscle weakness, tremor, or memory), and/or laboratory tests.

USP13 independently regulates parkin and alpha-synuclein ubiquitination. USP13 knockdown increases alpha-synuclein ubiquitination and clearance and regulates parkin function. USP13 knockdown also increases hyper-phosphorylated tau (p-tau) ubiquitination and facilitates the clearance of p-tau and amyloid-β (Aβ) peptides. Neurotoxic proteins including p-tau, Aβ, and alpha-synuclein co-exist in neurodegenerative diseases and may be simultaneously degraded by inhibiting USP13. Upon reduction of USP13 activity, p-tau, Aβ, and/or alpha-synuclein levels are also reduced. Therefore, the compounds described herein are also effective in reducing p-tau, Aβ, and/or alpha-synuclein levels in a cell. Optionally, the compounds described herein can cross the blood brain barrier.

The methods set forth herein optionally include administering a second therapeutic agent to the subject. For example, in order to treat a neurodegenerative disease, the second therapeutic agent can be selected from the group consisting of levadopa, a dopamine agonist, an anticholinergic agent, a monoamine oxidase inhibitor, a COMT inhibitor, amantadine, rivastigmine, an NMDA antagonist, a cholinesterase inhibitor, riluzole, an anti-psychotic agent, an antidepressant, and tetrabenazine. In another example, in order to treat cancer, the second therapeutic agent, can be, for example, a chemotherapeutic agent or radiation.

The compounds described herein can increase parkin activity in a subject. As used throughout, an increase in parkin activity in a subject can be an increase of about 10% , 20%, 30%, 40%, 50%, 60%, 70%, 80% 90%, 100% or greater as compared to a control. For example, the increase in parkin activity can be an increase of about 10%, 20%, 30%, 40%, 50%, 60%, 70%, 80% 90%, 100% or greater as compared to a subject that was not administered a compound as described herein or a control value.

The amount of inhibition or reduction of activity of USP13 does not have to be complete as this can range from a decrease to complete ablation of enzymatic activity. For example, the reduction can be about a 10%, 20%, 30%, 40%, 50%, 60%, 70%, 80% 90%, 100%, or any amount of reduction in between. Inhibition or reduction of activity of USP13 can be due to a decrease in mRNA expression, a decrease in protein expression, and/or a decrease in the enzymatic activity of USP13.

Further provided is a method of treating or preventing a neurodegenerative disease, a myodegenerative disease or prion disease in a subject, comprising administering to the subject an effective amount of a compound or pharmaceutical composition as described herein. The method can optionally include selecting a subject with a neurodegenerative disease of the central nervous system, a myodegenerative disease, a prion disease or at risk for a neurodegenerative disease of the central nervous system, a myodegenerative disease or a prion disease.

Also provided is a method of treating or preventing cancer in a subject, comprising administering to the subject an effective amount of a compound or pharmaceutical composition as described herein. The method can optionally include selecting a subject with cancer or at risk for cancer.

Modes of administration of the compositions used in the invention are exemplified below. The compounds described herein can be delivered by any of a variety of routes including by injection (e.g., subcutaneous, intramuscular, intravenous, intra-arterial, intraperitoneal), by continuous intravenous infusion, cutaneously, dermally, transdermally, orally (e.g., tablet, pill, liquid medicine, edible film strip), by implanted osmotic pumps, by suppository, or by aerosol spray. Routes of administration include, but are not limited to, topical, intradermal, intrathecal, intralesional, intratumoral, intrabladder, intravaginal, intraocular, intrarectal, intrapulmonary, intracranial, intraventricular, intraspinal, dermal, subdermal, intra-articular, placement within cavities of the body, nasal inhalation, pulmonary inhalation, impression into skin, and electroporation.

The compounds described herein are also useful in reducing alpha-synuclein levels in a cell. The methods for reducing alpha-synuclein levels in a cell include contacting a cell with an effective amount of one or more of the compounds as described herein. Optionally, the contacting is performed in vivo. Optionally, the contacting is performed in vitro.

V. Kits

Also provided herein are kits (also referred to as a pharmaceutical pack) for treating or preventing a USP13-related disease (e.g., a neurodegenerative disease or cancer) in a subject. The pharmaceutical pack or kit includes one or more containers filled with one or more of the ingredients of the pharmaceutical compositions. A kit can include any of the compounds or compositions described herein. For example, a kit can include one or more compounds of Formula I and/or Formula II. A kit can further include one or more additional agents, such as one or more of levadopa, a dopamine agonist, an anticholinergic agent, a monoamine oxidase inhibitor, a COMT inhibitor, amantadine, rivastigmine, an NMDA antagonist, a cholinesterase inhibitor, riluzole, an anti-psychotic agent, an antidepressant, tetrabenazine, or a chemotherapeutic agent. A kit can include an oral formulation of any of the compounds or compositions described herein. A kit can include an intravenous formulation of any of the compounds or compositions described herein. A kit can additionally include directions for use of the kit (e.g., instructions for treating a subject), a container, a means for administering the compounds or compositions (e.g., a syringe), and/or a carrier.

Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

As used throughout, treat, treating, and treatment refer to a method of reducing or delaying one or more effects or symptoms of a disease or disorder, for example, a neurodegenerative disease or cancer. The subject can be diagnosed with a disease or disorder. Treatment can also refer to a method of reducing the underlying pathology rather than just the symptoms. The effect of the administration to the subject can have the effect of, but is not limited to, reducing one or more symptoms of the disease or disorder, a reduction in the severity of the disease or disorder, the complete ablation of the disease or disorder, or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10%, 20%, 30%, 40%, 50%, 60%, 70%, 80%, 90%, 100%, or any amount of reduction in between.

As utilized herein, by prevent, preventing, or prevention is meant a method of precluding, delaying, averting, obviating, forestalling, stopping, or hindering the onset, incidence, severity, or recurrence of the neurodegenerative disease or disorder. For example, the disclosed method is considered to be a prevention if there is a reduction or delay in onset, incidence, severity, or recurrence of neurodegeneration or one or more symptoms of neurodegeneration (e.g., tremor, weakness, memory loss, rigidity, spasticity, atrophy) in a subject susceptible to neurodegeneration as compared to control subjects susceptible to neurodegeneration that did not receive a USP13 inhibitor as described herein. The disclosed method is also considered to be a prevention if there is a reduction or delay in onset, incidence, severity, or recurrence of neurodegeneration or one or more symptoms of neurodegeneration in a subject susceptible to neurodegeneration after receiving a USP13 inhibitor as compared to the subject's progression prior to receiving treatment. Thus, the reduction or delay in onset, incidence, severity, or recurrence of neurodegeneration can be about a 10%, 20%, 30%, 40%, 50%, 60%, 70%, 80%, 90%, 100%, or any amount of reduction in between.

As used throughout, by subject is meant an individual. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the subject matter described herein which are apparent to one skilled in the art.

### Example 1: Synthesis of USP-13 Inhibitors

### General Information

Commercially available 7-chlorothieno[3,2-b]pyridine (1), aniline (2), 4-chloro-6-fluoroquinoline (3), benzyl alcohol (4), benzyl amine (5), 4-chloro-3-nitro-2H-chromen-2-one (6), reagents and solvents were used as purchased without further purification. Methyl (S)-phenylalaninate (7) and methyl (S)-tyrosinate (8) were obtained by means of extraction with aqueous NaHCO₃ and EtOAc from their HCl salts. NMR spectra were obtained at 400 MHz (¹H NMR) and 100 MHz (¹³C NMR) in deuterated solvents. Reaction products were purified by column chromatography on silica gel (particle size 40-63 μm) as described below.

### Synthetic Methods and Compound Characterization

Compounds BK50118-A, BK50118-B, and BK50118-C were synthesized according to Scheme 1 shown below.

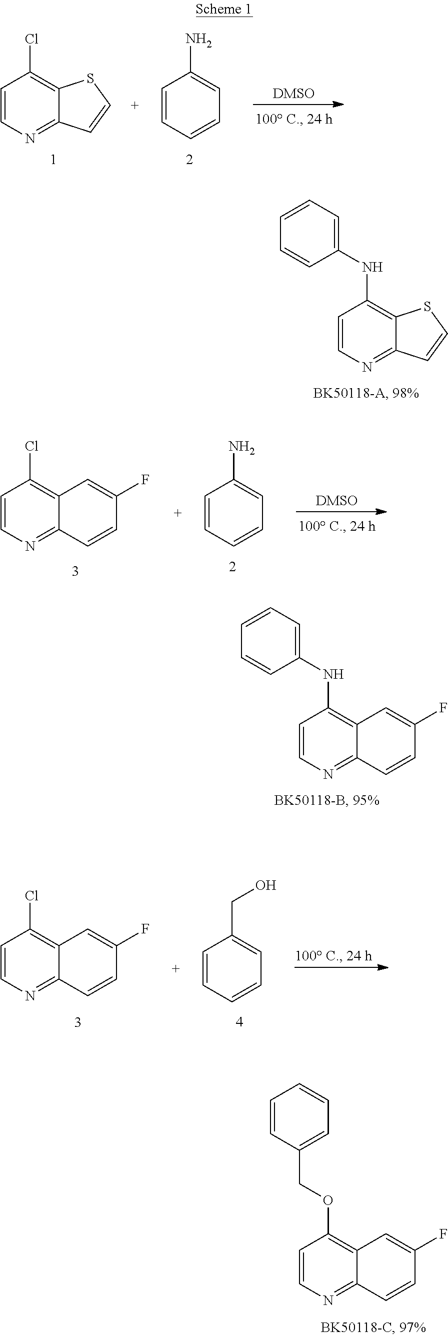

A general procedure for preparing the compounds according to Scheme 1 is provided below: An 8 mL pressure vessel was charged with 7-chlorothieno[3,2-b]pyridine (1) (0.6 mmol), aniline (1.2 mmol) and DMSO (1.0 mL) unless noted otherwise. The pressure vessel was then placed in a 100° C. oil bath and the mixture was stirred for 24 hours. After full conversion was achieved based on ¹H NMR analysis, the reaction mixture was extracted with EtOAc. The combined organic layers were extracted with water, dried over sodium sulfate and the solvent was removed in vacuo. The crude product was purified by flash chromatography on silica gel using with hexanes-ethyl acetate as mobile phase as described below.

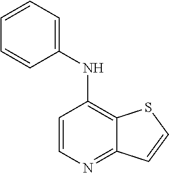 [SMILES: c1ccc(Nc2ccnc3ccsc23)cc1]

N-Phenylthieno[3,2-b]pyridin-7-amine (BK50118-A). Compound BK50118-A was obtained as a colorless solid in 98% yield (133 mg, 0.59 mmol) from 7-chlorothieno[3,2-b]pyridine (101 mg, 0.6 mmol) and aniline (111 mg, 1.2 mmol) in 1 mL of DMSO after 24 hours at 100° C. by following the general procedure described above. The crude product was purified using flash chromatography on silica gel using hexanes/EtOAc (1:1) as mobile phase. R_{f}=0.3 (hexanes/EtOAc, 1:1); 1 HNMR (400 MHz, Chloroform-d) δ=8.42 (d, J=5.4 Hz, 1H), 7.63 (d, J=5.4 Hz, 1H), 7.53 (d, J=5.4 Hz, 1H), 7.45-7.37 (m, 2H), 7.32-7.25 (m, 2H), 7.19 (m, 1H), 6.92 (d, J=5.4 Hz, 1H), 6.12 (s, 1H); ¹³C NMR (100 MHz, Chloroform-d) δ=157.8, 148.9, 146.3, 139.4, 129.7, 128.1, 126.5, 124.9, 122.6, 120.8, 102.3.

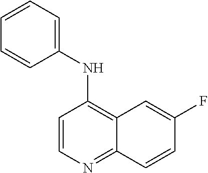 [SMILES: Fc1ccc2nccc(Nc3ccccc3)c2c1]

6-Fluoro-N-phenylquinolin-4-amine (BK50118-B). Compound BK50118-B was obtained as a colorless solid in 95% yield (135 mg, 0.57 mmol) from 4-chloro-6-fluoroquinoline (109 mg, 0.6 mmol) and aniline (111 mg, 1.2 mmol) in 1 mL of DMSO after 24 hours at 100° C. by following the general procedure described above. The crude product was purified using flash chromatography on silica gel using hexanes/EtOAc (1:1) as mobile phase. R_{f}=0.3 (hexanes/EtOAc, 1:1); ¹H NMR (400 MHz, Chloroform-d) δ=8.54 (d, J=5.2 Hz, 1H), 8.04 (dd, J=9.9, 5.2 Hz, 1H), 7.58 (dd, J=9.9, 2.7 Hz, 1H), 7.49-7.37 (m, 3H), 7.28 (d, J=7.7 Hz, 2H), 7.19 (m, 1H), 7.01 (d, J=5.2 Hz, 1H), 6.59 (s, 1H); 13 C NMR (100 MHz, Chloroform-d) δ=160.2 (d, J=246.7 Hz), 150.3 (d, J=2.3 Hz), 147.3 (d, J=5.0 Hz), 146.4, 139.9, 132.8 (d, J=9.0 Hz), 129.9, 124.9, 122.7, 120.5 (d, J=8.5 Hz), 119.4 (d, J=25.1 Hz), 104.1 (d, J=23.1 Hz), 103.0; ¹⁹F NMR (376 MHz, Chloroform-d) δ=−114.04 (m).

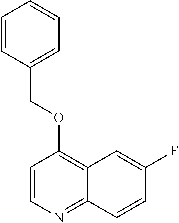 [SMILES: Fc1ccc2nccc(OCc3ccccc3)c2c1]

4-(Benzyloxy)-6-fluoroquinoline (BK50118-C). Compound BK50118-C was obtained as a colorless solid in 97% yield (147 mg, 0.58 mmol) from 4-chloro-6-fluoroquinoline (109 mg, 0.6 mmol) and benzyl alcohol (1 mL) after 16 hours at 100° C. by following the general procedure described above. The crude product was purified using flash chromatography on silica gel using hexanes/EtOAc (1:1) as mobile phase. R_{f}=0.5 (hexanes/EtOAc, 1:1); ¹H NMR (400 MHz, Chloroform-d) δ=8.70 (d, J=5.2 Hz, 1H), 8.03 (dd, J=9.5, 5.2 Hz, 1H), 7.85 (dd, J=9.5, 2.9 Hz, 1H), 7.54-7.33 (m, 6H), 6.80 (d, J=5.2 Hz, 1H), 5.27 (s, 2H); ¹³C NMR (100 MHz, Chloroform-d) δ=160.8 (d, J=5.1 Hz), 160.2 (d, J=246.7 Hz), 150.6 (d, J=2.5 Hz), 146.4 (d, J=1.2 Hz), 135.5, 131.5 (d, J=9.0 Hz), 128.9, 128.6, 127.5, 122.2 (d, J=9.6 Hz), 119.9 (d, J=25.6 Hz), 105.9 (d, J=23.4 Hz), 101.7, 70.5; ¹⁹F NMR (376 MHz, Chloroform-d) δ=−113.82 (m).

Compound CL3-499 was synthesized according to Scheme 2 shown below and the following procedure.

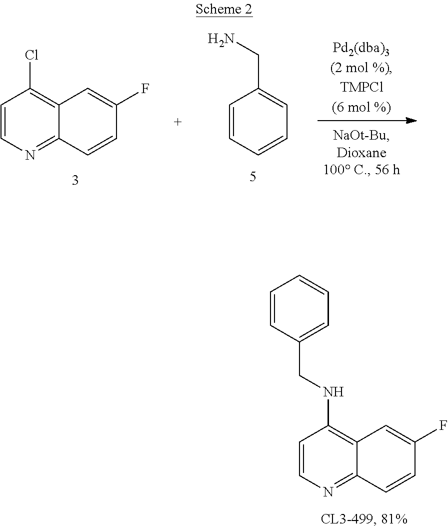 [SMILES: Fc1ccc2nccc(Cl)c2c1 ; NCc1ccccc1 ; Fc1ccc2nccc(NCc3ccccc3)c2c1]

An 8 mL sealed vial was charged with 4-chloro-6-fluoroquinoline (3) (100 mg, 0.55 mmol), Pd₂(dba)₃ (10.1 mg, 0.01 mmol, 2 mol %), 1,3-bis(2,4,6-trimethylphenyl)imidazolium chloride (TMPC1) (11.3 mg, 0.03 mmol, 6 mol %), NaOt-Bu (169.2 mg, 1.76 mmol), benzylamine (5) (177 mg, 1.65 mmol) and dioxane (1.5 mL). The vial was then placed in a 100° C. oil bath and stirred for 56 hours. The reaction was quenched with aqueous NaHCO₃ and extracted with EtOAc. The combined organic layers were dried over sodium sulfate and the solvent was removed in vacuo. The crude product was purified by flash chromatography on silica gel using hexanes-EtOAc (8:2) as mobile phase and recrystallized from hexanes-EtOAc (1:1).

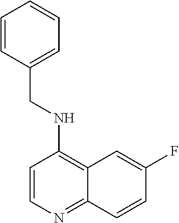 [SMILES: Fc1ccc2nccc(NCc3ccccc3)c2c1]

N-Benzyl-6-fluoroquinolin-4-amine (CL3-499). Compound CL3-499 was obtained as a yellow solid in 81% yield (112 mg, 0.45 mmol). R_{f}=0.16 (hexanes/EtOAc, 1:1); ¹H NMR (400 MHz, Chloroform-d) δ 8.51 (d, J=5.2 Hz, 1H), 7.99 (dd, J=5.8 Hz, J=5.3 Hz, 1H), 7.43-7.31 (m, 7H), 6.47 (d, J=5.2 Hz, 1H), 5.18 (bs, 1H), 4.53 (d, J=5.2 Hz, 2H); ¹³C NMR (100 MHz, Chloroform-d) δ 159.7 (d, J=245.0 Hz), 150.4 (d, J=2.3 Hz), 149.0 (d, J=4.7 Hz), 145.5, 137.3, 132.4 (d, J=8.9 Hz), 128.9, 127.9, 127.5, 119.2 (d, J=8.6 Hz), 118.8 (d, J=24.9 Hz), 103.7 (d, J=22.6 Hz), 99.8, 47.6; 19F NMR (376 MHz, Chloroform-d) δ −114.97 (m).

Compounds CL3-512 and CL3-514 were synthesized according to Scheme 3 shown below.

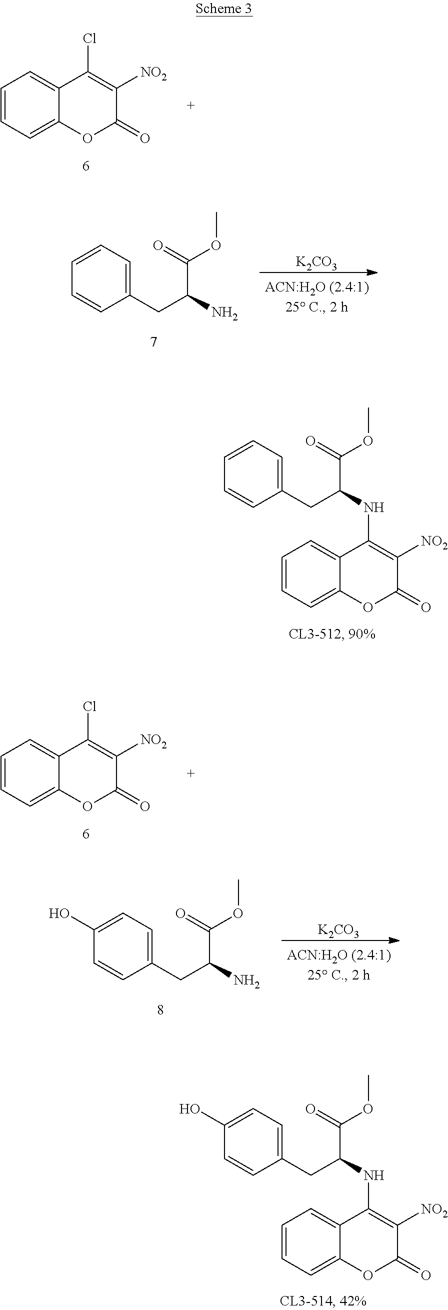

A general procedure for preparing the compounds according to Scheme 3 is provided below: An 8 mL vial was charged with 4-chloro-3-nitro-2H-chromen-2-one (6), amino ester derivative (7) or (8), K₂CO₃ and ACN:H₂O (6.8 mL, 2.4:1). The reaction mixture was stirred at 25° C. for 2 hours. After full conversion was achieved based on TLC analysis, the pH was adjusted to 7 and the reaction mixture was extracted with EtOAc. The combined organic layers were washed with water, dried over sodium sulfate and the solvent was removed in vacuo. The crude product was purified by flash chromatography on silica gel as described below.

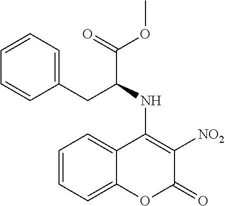 [SMILES: COC(=O)[C@H](Cc1ccccc1)Nc1c([N+](=O)[O-])c(=O)oc2ccccc12]

(S)-Methyl (3-nitro-2-oxo-2H-chromen-4-yl)-phenylalaninate (CL3-512). Compound CL3-512 was obtained after column purification using DCM/MeOH (9:1) as mobile phase as a colorless solid in 90% yield (274 mg, 0.8 mmol) from 4-chloro-3-nitro-2H-chromen-2-one (6) (200 mg, 0.89 mmol), methyl (S)-phenylalaninate (7) (146.5 mg, 0.89 mmol) and K₂CO₃ (246 mg, 1.78 mmol) by following the general procedure described above. R_{f}=0.44 (DCM/MeOH, 9:1); ¹H NMR (400 MHz, Methanol-d₄) δ 7.98 (d, J=8.3 Hz, 1H), 7.67 (dd, J=9.1 Hz, 7.2 Hz, 1H), 7.37 (dd, J=9.0 Hz, 7.3 Hz, 1H), 7.28 (d, J=8.3 Hz, 1H), 7.21-7.13 (m, 5H), 4.57 (bs, 1H), 3.34 (dd, J=14.1 Hz, 4.5 Hz), 3.29 (s, 3H), 3.22 (dd, J=14.1 Hz, 8.7 Hz); ¹³C NMR (100 MHz, Methanol-d₄) δ 172.1, 154.9, 151.0, 135.8, 133.8, 128.7, 128.3, 126.9, 124.5, 123.5, 117.2, 116.7, 113.1, 58.8, 53.4, 38.1.

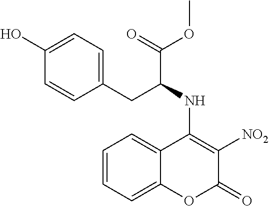 [SMILES: COC(=O)[C@H](Cc1ccc(O)cc1)Nc1c([N+](=O)[O-])c(=O)oc2ccccc12]

(S)-Methyl (3-nitro-2-oxo-2H-chromen-4-yl)-tyrosinate (CL3-514). Compound CL3-514 was obtained after column purification using DCM/MeOH (9:1) as mobile phase as a colorless solid in 42% yield (137.1 mg, 0.37 mmol) from 4-chloro-3-nitro-2H-chromen-2-one (6) (200 mg, 0.89 mmol), methyl (S)-tyrosinate (8) (161 mg, 0.89 mmol) and K₂CO₃ (246 mg, 1.78 mmol) by following the general procedure described above. R_{f}=0.21 (DCM/MeOH, 9:1); ¹H NMR (400 MHz, Methanol-d₄) δ 7.78 (d, J=8.2 Hz, 1H), 7.55 (dd, J=8.5 Hz, 8.5 Hz, 1H), 7.24 (dd, J=8.5 Hz, 8.4 Hz, 1H), 7.18 (d, J=8.2 Hz, 1H), 6.81 (d, J=8.1 Hz, 2H), 6.45 (d, J=8.1 Hz, 2H), 4.40 (bs, 1H), 3.25 (s, 3H), 3.16 (dd, J=14.1 Hz, 4.5 Hz, 1H), 3.04 (dd, J=14.1 Hz, 8.5 Hz, 1H); ¹³ C NMR (100 MHz, Methanol-d₄) δ 173.8, 156.1, 150.5, 135.9, 130.1, 126.3, 123.9, 117.4, 116.0, 114.8, 113.7, 48.4, 38.0, 29.9.

### Example 2: Activity Assays

Cell lines, transfection, and treatment. Human SHSY-5Y neuroblastoma cells were cultured in Dulbecco's Modified Eagle's Medium (DMEM) (ThermoFisher Scientific; Waltham, MA) with 10% Fetal Bovine Serum (FBS) (ThermoFisher) and 1% Penicillin-Streptomycin (PenStrep, ThermoFisher) and incubated at 37° C. under atmospheric oxygen concentrations (21%) with 5% CO₂. SHSY-5Y cells were cultured in DMEM with Ham's F12 (1:1) (ThermoFisher) with 20% FBS, 1% PenStrep. Cells were plated at a density tailored to reach 70-80% confluence at the beginning of every experiment. SH-SY5Y cells were transiently transfected for 24 hours with human wild-type α-synuclein using Fugene HD transfection reagent (Promega; Madison, WI) before fresh culture media and drug were added. Cells were treated with 1 mM, 100 μM, 10 μM, 1 μM, 0.1 μM, and 0.01 μM of BK50118-A, BK50118-B, BK50118-C, CL3-499, CL3-512 and CL3-514 dissolved in DMSO or an equivalent volume of DMSO for 5 hours.

Cell viability was determined via lactate dehydrogenase assay on culture media, (ThermoFisher) and MTT assay on plated cells (ThermoFisher). Using separate samples with the same treatment conditions, cells were harvested on ice by removing culture medium and adding 0.2 ml 1× sodium-tris, EDTA, NP-40 (STEN) buffer (50 mM Tris (pH 7.6), 150 mM NaCl, 2 mM EDTA, 0.2% NP-40, 0.2% with Halt protease and phosphatase inhibitor solution (ThermoFisher). Cells were detached with a cell scraper and collected into centrifuge tubes and incubated at 4° C. for 30 minutes with agitation. Samples were stored at −80° C. and used for additional analyses.

Alpha-Synuclein Enzyme-Linked Immunosorbent Assay (ELISA). ELISAs for total human alpha-synuclein (Millipore; Burlington, MA) were conducted using Milliplexed ELISA. Briefly, Xmap technology uses magnetic microspheres that are internally coded with two fluorescent dyes. Through precise combinations of these two dyes, multiple proteins are simultaneously measured within a sample. Each of these spheres is coated with a specific capture antibody. The capture antibody binds to the detection antibody and a reporter molecule, completing the reaction on the surface of the bead.

A total of 25 μL soluble protein was incubated overnight at 4° C. with 25 μL of a mixed bead solution. After washing, samples were incubated with 25 μL detection antibody solution for 1.5 hours at room temperature. Streptavidin-phycoerythrin (25 μL) was added to each well containing the 25 μL of detection antibody solution. Samples were then washed and suspended in 100 μL of sheath fluid. Samples were then run on MAGPIX with Xponent software. The median fluorescent intensity (MFI) data were analyzed using a 5-parameter logistic or spline curve-fitting method for calculating analyte concentrations in samples. Specific alpha-synuclein ELISAs were performed on tissue soluble extracts from midbrain lysates in 1XSTEN buffer. The ELISA for USP13 (MyBioSource, Cat # MBS9335287), Human alpha-synuclein (Biolegend, Cat #844101) and specific p-Tau ser396 (Invitrogen, KHB7031) were performed on cell or brain tissue extracts as described above according to manufacturers' protocol.

Western Blot Analysis. To extract the soluble proteins from mouse midbrain lysates, tissues were isolated and homogenized in 1×STEN buffer (50 mM Tris (pH 7.6), 150 mM NaCl, 2 mM EDTA, 0.2% NP-40, 0.2% BSA, 20 mMPMSF and protease cocktail inhibitor), centrifuged at 10,000×g for 20 min at 4° C., and the supernatant containing the soluble protein fraction was collected. Extracts were analyzed by Western blot (WB) on 4-12% SDS NuPAGE Bis-Tris gel (Invitrogen, NP0301BOX). Beta-actin (β-actin) was probed (1:3000) with monoclonal antibody (Emdmillipore, MAB1501R). Human alpha-synuclein was probed (1:2000) with monoclonal antibody (Thermo Fisher, AHB0261, Rockford, IL, USA). USP13 was probed (1:1000) with polyclonal antibody (ThermoFisher, PAS-12014, Rockford, IL, USA). Ubiquitin was probed (1:5000) with polyclonal antibody (Thermo Fisher, PA3-16717, Rockford, IL, USA). WBs were quantified by densitometry using Quantity One 4.6.3 software (Bio Rad, Hercules, CA, USA) and Image J.

Immunoprecipitation (IP). Mouse brain tissues were homogenized in 1×STEN buffer, and the soluble fraction was isolated as indicated above. The lysates were pre-cleaned with immobilized recombinant protein A/G agarose (Santa Crutz, sc-2003, Dallas, TX, USA) and centrifuged at 2500×g for 1 min at 4° C. The supernatant was recovered and quantified by protein assay, and a total of 300 μg protein was incubated overnight at 4° C. with primary anti-alpha-synuclein (1:200, Thermofisher, AHB0261) mouse antibodies or anti-ubiquitin (1:100) (Thermo Fisher, PA3-16717, Rockford, IL, USA) antibodies in the presence of sepharose G and an IgG control with primary antibodies. The immunoprecipitates were collected by centrifugation at 2500×g for 3 min at 4° C., washed 5× in PBS, with spins of 3 min, 2500×g using detergent-free buffer for the last washing step, and the proteins were eluted according to Pierce instructions (Pierce #20365, Rockford, IL, USA). After IP, the samples were size-fractionated on 4-12% SDS-NuPAGE and transferred onto 0.45 μm nitrocellulose membranes. WB detection was then performed using horseradish peroxidase (HRP)-conjugated secondary antibodies.

Immunohistology. Animals were deeply anesthetized with a mixture of xylazine and ketamine (1:8), washed with normal saline for 1 min and then perfused with 4% paraformaldehyde (PFA) for 15-20 min. Brains were quickly dissected out and immediately stored in 4% PFA for 24 h at 4° C. and then transferred to 30% sucrose at 4° C. for 48 h. Brains were cut using a cryostat microtome into 20 μm thick coronal sections and stored at −20° C.

Immunohistochemistry was performed on the 20 μm thick brain sections for evaluation of ubiquitination of alpha-synuclein. The antibodies used were human alpha-synuclein monoclonal antibody (Thermo Fisher, AHB0261, Rockford, IL, USA) and ubiquitin polyclonal antibody (Thermo Fisher, PA3-16717, Rockford, IL, USA). The optic densitometry of co-localization of ubiquitin with alpha-synuclein was measured using Image J. Tyrosine hydroxylase (TH) is the limiting enzyme in DA synthesis, so probing for TH+ neurons will help to evaluate the status of DA producing neurons in the SN and their terminals in the striatum. We performed fluorescent staining of TH+ in striatum and conducted optic densitometry measurement of striatal DA terminals. Nuclear staining with 4,6-diamidino-2-phenylindole was performed according to manufacturer's protocols (Life Technologies, Rockford, IL, USA).

DAB staining was performed for alpha-synuclein (Thermo Fisher, AHB0261, Rockford, IL, USA) on the 20 μm thick mouse brain sections, and stereological counting of alpha-synuclein+ neurons counterstained with Nissl was conducted.

Nissl and Silver Staining. Nissl staining was performed using FD Cresyl Violet Solution™ Regular Strength (FD NeuroTechnologies, Cat PS102-01) as per manufacturer's instructions. Silver staining was performed using FD NeuroSilver™ (FD NeuroTechnologies, Cat PK301,) as per manufacturer's instructions.

Pharmacokinetics Studies. C57BL/6 mice received a single intraperitoneal dose (10 mg/kg) of BK50118-C. Brain and serum samples were collected at 1, 2, 3, 4, 8 and 12 h (n=3 per time point). Animals injected with vehicle (DMSO) were used for background subtraction. Stock solution of the drug (1 mg/mL) and internal standards were prepared in methanol. Intermediate solutions used for the calibrators and control samples were serial-diluted in methanol/water (1:1). Preparation of the calibration curve standards and quality samples (QC) were performed by mixing the intermediate dilutions in blank samples (brain homogenates, serum). The internal standard working solution contained deuterium labeled BK50118-C-d7 at the concentration of 5 ng/mL diluted in acetonitrile (ACN)/ethyl acetate (4:1). Serum and brain samples were stored at −80° C. and then thawed to room temperature prior to preparation. The brains were homogenized in MilliQ water (1 mg brain: 10 μL water). Proteins were precipitated in both brain and serum samples by mixing 25 μL aqueous sample with 75 μL internal standard working solution. The mixture was centrifuged at 12,300 g for 5 min. Thereafter, 75 μL of each supernatant and 25 μL of MilliQ water were pipetted into a 96-well PCR plate (Fisher Scientific, Dawsonville, GA,USA).

The concentrations of BK50118-C in the brain tissue and serum samples were measured by ultrahigh performance liquid chromatography tandem mass spectrometry (UHPLC-MS/MS). Briefly, the UHPLC-MS/MS system included an Elute HTG binary gradient UHPLC pump, an Elute column oven and an EVOQ Elite triple quadrupole mass spectrometer (all from Bruker Daltonik GmbH, Bremen, Germany) equipped with an electrospray ionization (ESI) source operating in a positive mode. The samples were injected by use of a PAL auto sampler (CTC Analytics, Zwingen, Switzerland) equipped with a 10-μL sample loop; the samples were kept in a PAL stack cooler for 6 microtiter plates and operating at +6° C. The system was controlled by a Compass 2.0/HyStar 4.0 software (Bruker); the compound screening and quantitation was performed by a TASQ 2.2 data acquisition and processing software (Bruker). The mass spectrometer was supplied by nitrogen and air generated by a Genius 3045 nitrogen/air generator (Peak Scientific Instruments, Inchinnan, Scotland, UK). The ESI parameters were as follows: probe gas flow 50, nebulizer gas flow 60, probe temperature +400° C., cone gas flow 20, cone temperature +350° C., CID gas Ar 1.5 mTorr. The mass spectra were scanned in the MRM mode to find the optimal collision energies for the test compounds and their respective precursor ions.

For chromatographic separation, an YMC-Ultra HT Hydrosphere C18 column (2.0×100 mm, 2 μm particle size) and an YMC-Hydrosphere 2.1×5 mm guard column (YMC Co. Ltd., Kyoto, Japan) were used. The mobile phase A was 10 mM ammonium formate, pH 4.3, in water; the mobile phase B was 10 mM ammonium formate, pH 4.3, in 10% water, 50% acetonitrile and 40% isopropyl alcohol. The mobile phase gradient was as follows (min—A/B%): 0 min—50/50, 0.5 min—50/50, 2.0 min—20/80, 3 min—20/80, 3.1 min—50/50, 4.5 min—50/50. The flow rate was 450 μL/min, and the column temperature was set at 50° C. The sample injection volume was 10 μL.

Statistical and Data Analysis. All statistical analyses were performed using GraphPad Prism version 5.0 (GraphPad software, Inc, San Diego, CA, USA). Two-tailed student's t-test was used in comparison of means of two groups. Ordinary one-way analysis of variance (ANOVA) followed by Newman-Keuls or Dunnett comparison post hoc tests were used in comparison of means of multiple groups. Asterisks denote actual p-value significances (*<0.05, **<0.01, ***<0.001 and ****<0.0001), and N is the number of animals or the number of independent experiments (cell culture) per group. Unless otherwise indicated, data are expressed as Mean±SD. IC50 to USP13 levels was also estimated using GraphPad Prism version 5.0 (GraphPad software, Inc, San Diego, CA, USA).

### Results:

Viability and activity assays of human SHSY5Y neuroblastoma cells treated with varying amounts of compounds as described herein were performed. Specifically, human SHSY5Y neuroblastoma cells were treated with 1 mM, 100 μM, 10 μM, 1 μM, 0.1 μM, and 0.01 μM of BK50118-A, BK50118-B, BK50118-C, CL3-499, CL3-512 and CL3514 dissolved in DMSO or an equivalent volume of DMSO for 5 hours.

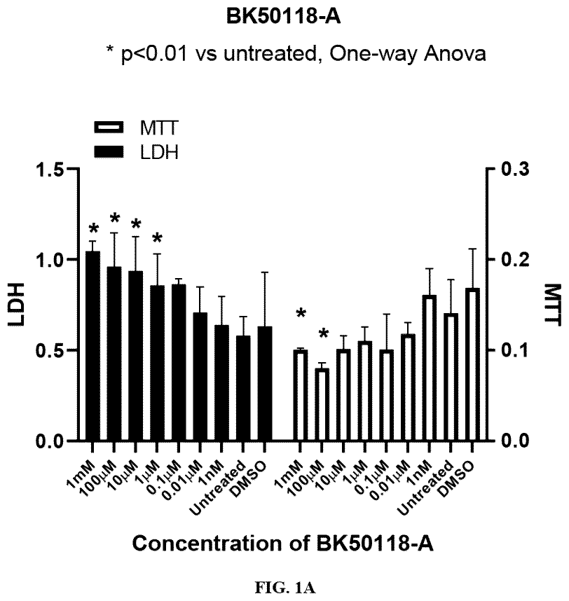

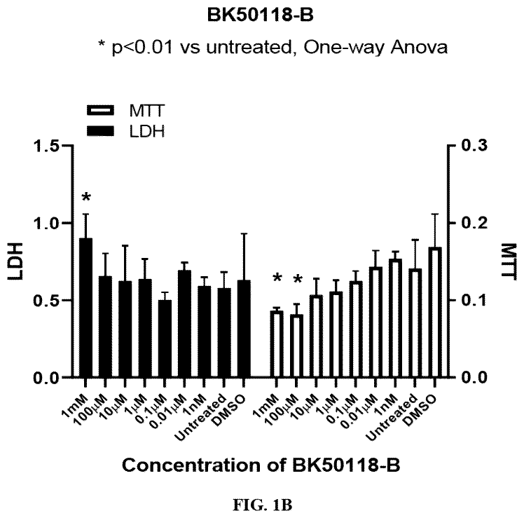

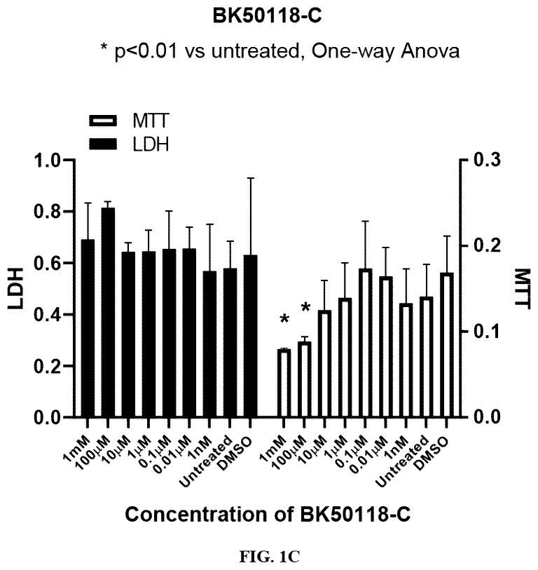

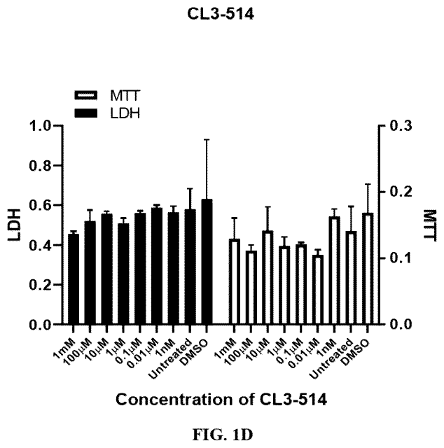

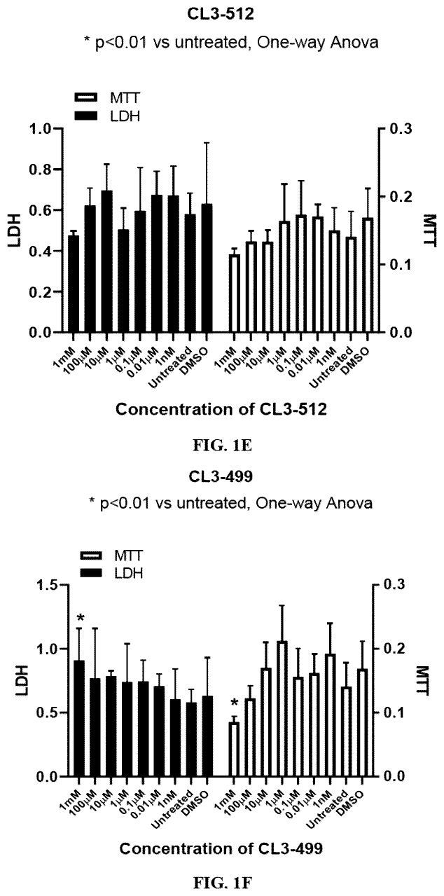

The graphs in FIGS. 1A-1F represent MTT and LDH assays in cells treated with BK50118-A (FIG. 1A), BK50118-B (FIG. 1B), BK50118-C (FIG. 1C), CL3-514 (FIG. 1D), CL3-512 (FIG. 1E) and CL3-499 (FIG. 1F). N=3-4 per group. The data in FIGS. 1A-1F indicate that compounds BK50118-A, BK50118-B, BK50118-C, CL3-514, CL3-512 and CL3-499 are safe and do not cause cell death using two viability assays up to 1 mM concentration.

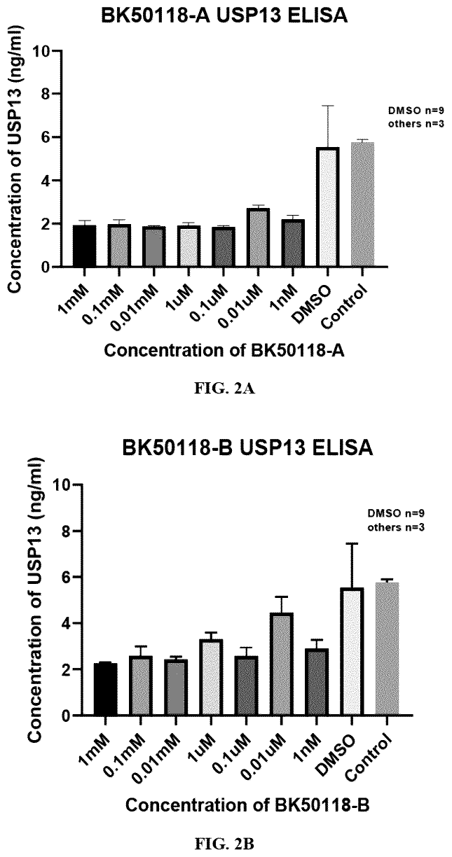

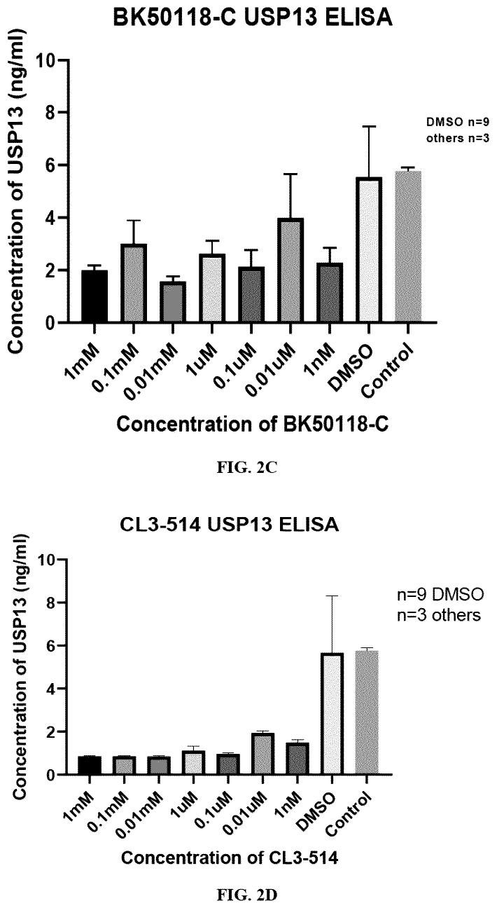

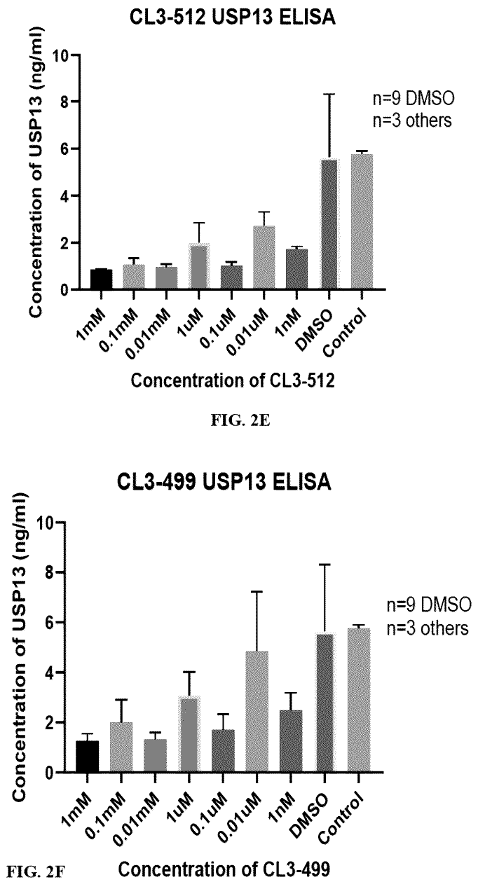

The graphs in FIGS. 2A-2F represent the USP13 activity via ELISA in cells treated with BK50118-A (FIG. 2A), BK50118-B (FIG. 2B), BK50118-C (FIG. 2C), CL3-514 (FIG. 2D), CL3-512 (FIG. 2E) and CL3-499 (FIG. 2F). N=3-9 per group. The data in FIGS. 2A-2F indicate that compounds BK50118-A, BK50118-B, BK50118-C, CL3-514, CL3-512 and CL3-499 significantly reduce the activity of human USP13 within a concentration range of 1 nM-1 mM concentration.

The half estimated maximal inhibition concentration (IC₅₀) of each compound needed to inhibit USP13 activity in the SHSY5Y cell line was determined (Table 1). Spautin-1 was also assessed for comparative purposes.

TABLE 1

| Compound Name | IC₅₀ | |
|---|---|---|
| BK50118-A | 26.0 | nM |
| BK50118-B | 36.9 | nM |
| BK50118-C | 78.0 | nM |
| CL3-514 | 68.9 | nM |
| CL3-512 | 1.08 | μM |
| CLS-499 | 1.19 | μM |
| Spautin-1 | 0.6-0.7 | μM |

The data in Table 1 indicate that compounds BK50118-A, BK50118-B, BK50118-C, CL3-514, CL3-512, and CL3-499 potently inhibit USP-13 activity.

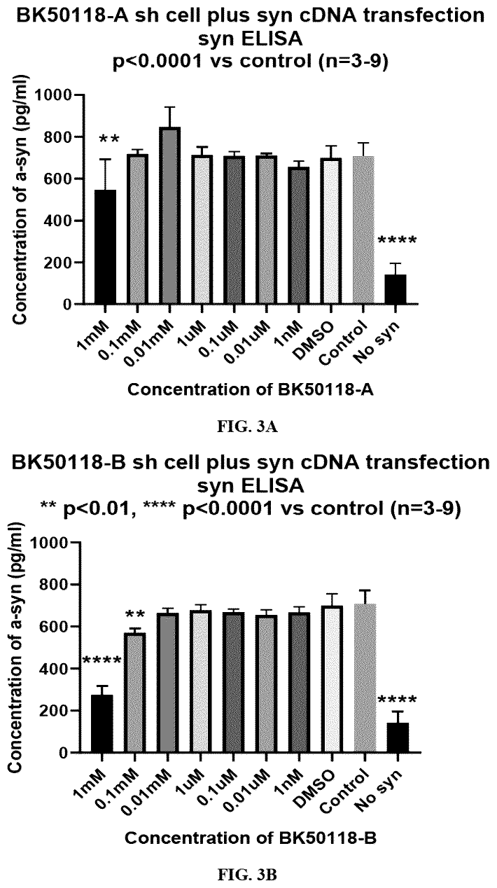

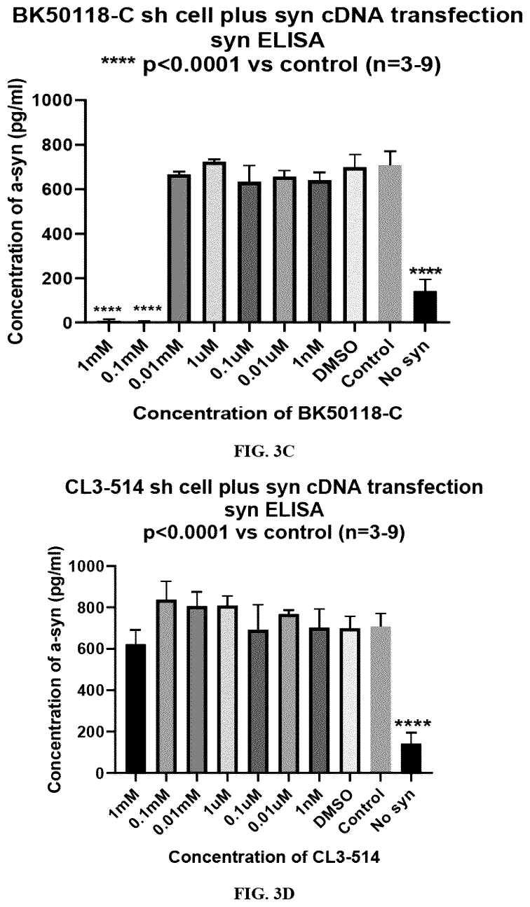

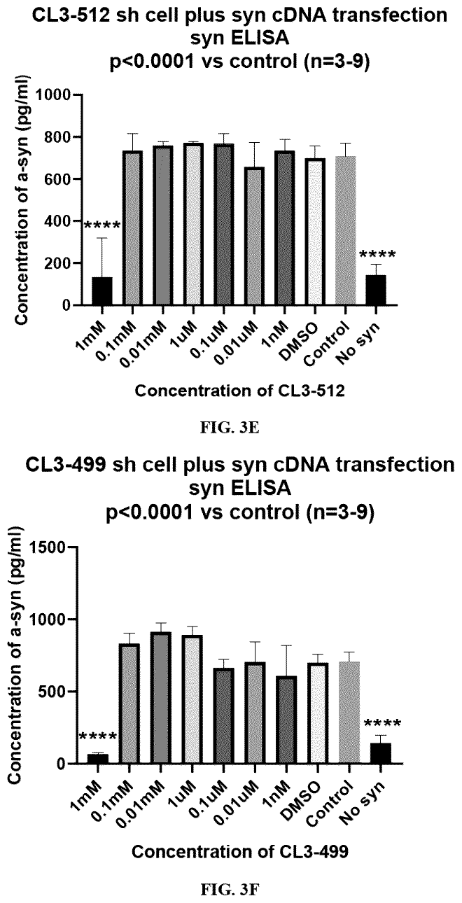

The graphs in FIGS. 3A-3F depict the alpha-synuclein levels via ELISA in human SHSY5Y neuroblastoma cells treated with 1 mM, 100 μM, 10 μM, 1 μM, 0.1 μM, and 0.01 μM of BK50118-A, BK50118-B, BK50118-C, CL3-499, CL3-512, and CL3-514 dissolved in DMSO or an equivalent volume of DMSO for 5 hours. The graphs in FIGS. 3A-3F represent the ELISA levels of alpha-synuclein in cells treated with BK50118-A (FIG. 3A), BK50118-B (FIG. 3B), BK50118-C (FIG. 3C), CL3-514 (FIG. 3D), CL3-512 (FIG. 3E) and CL3-499 (FIG. 3F). N=3-9 per group. The data in FIGS. 3A-3F indicate that compounds BK50118-A, BK50118-B, BK50118-C, CL3-514, CL3-512 and CL3-499 significantly reduce the level of alpha-synuclein with a concentration between 1 and 0.1 nM.

In Vivo studies

Since BK50118-C is a potent USP13 inhibitor, the pharmacokinetics (PK) of this molecule were determined. Wild type C57BL6 mice were injected with 10 mg/kg BK50118-C versus DMSO, and the brain and serum were isolated at 0, 1, 2, 4, 6, 8 and 12 h, extracted in water and examined by mass spectrometry. The concentration of BK50118-C peaked at 1 h (Tₘₐₓ) both in the serum and brain (Table 2), and a maximal concentration (Cₘₐₓ) of 81.49 nM and 354.63 nM were reached in the brain and serum, respectively. The bioavailabilities of the drug (AUC, area under the curve) were 164.3 nM×hand 599.4 nM×h in the brain and serum, respectively. Elimination (T_{1/2}) was 2.32 h for brain and 1.84 h in serum. The ratio of serum: brain reached 28%, indicating that this drug abundantly enters the brain.

TABLE 2
Pharmacological parameters of BK50118-C measured in wild type
mice (C57B6) injected with 10 mg/kg of deuterated BK50118-C
and tissues were collected over 12 h after injection.

| | | |
|---|---|---|
| BK50118-C | Dosage (mg/kg, I.P) | 10.00 |
| | Total drug injected (nmol) | 1185.77 |
| Brain | Cₘₐₓ (nM) | 81.49 ± 69.97 |
| | Cₘₐₓ (ng/mL) | 20.62 ± 17.70 |
| | Tₘₐₓ (h) | 1.00 |
| | AUC (nM · h) | 164.3 ± 39.49 |
| | T_{1/2} (elimination) (h) | 2.32 |
| Serum | Dosage (mg/Kg) | 10.00 |
| | Cₘₐₓ (nM) | 354.63 ± 272 |
| | Cₘₐₓ (ng/mL) | 89.72 ± 68.81 |
| | Tₘₐₓ (h) | 1.00 |
| | AUC (nM · h) | 599.4 ± 142.9 |
| | T_{1/2} (elimination) (h) | 1.84 |
| | Ratio of Serum/Brain (%) | 28.0% |

### BK50118-C Reduces Alpha-Synuclein, Increases Alpha-Synuclein Ubiquitination and Improves Neuronal Survival in Mice

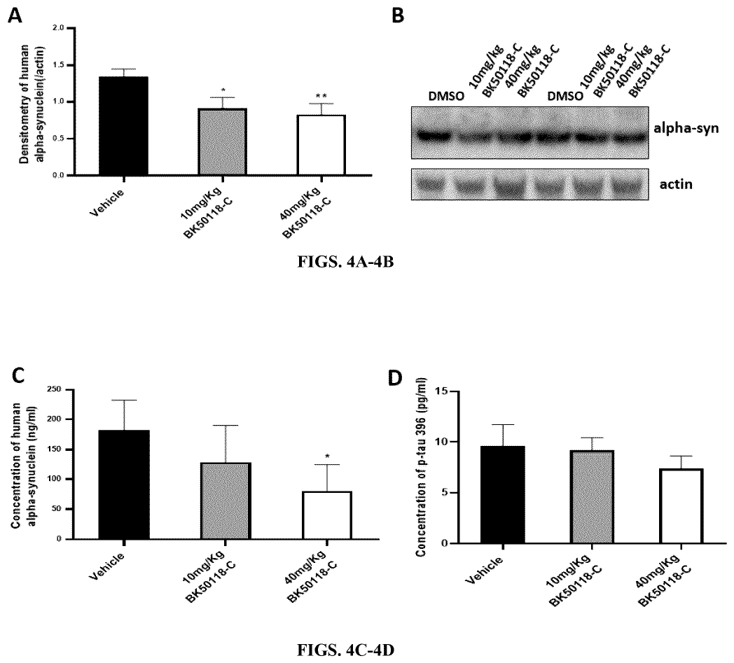

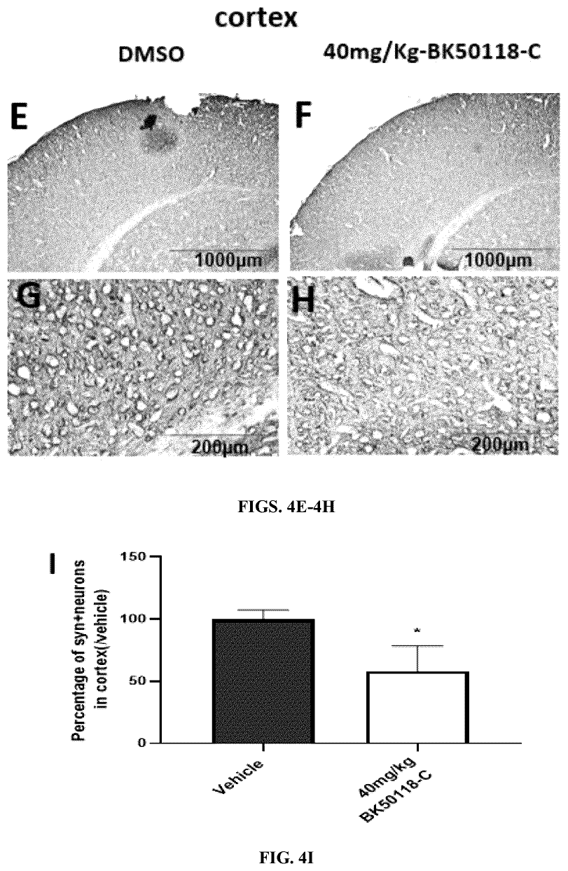

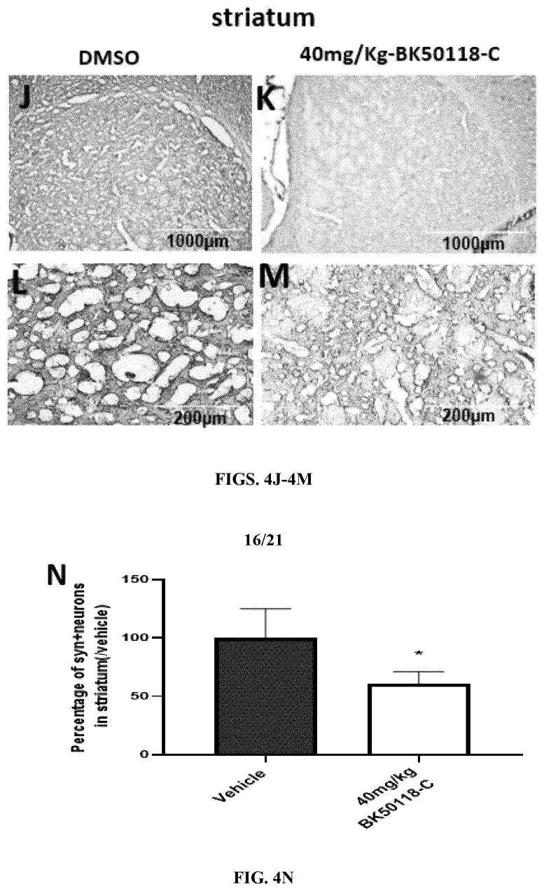

Transgenic A53T mice harbor the arginine to threonine (A53T) mutation of human alpha-synuclein under the control of prion promoter and have abundant alpha-synuclein in the striatum as early as 3 months of age. Male and female TgA53T mice (15 months old) were treated daily with intraperitoneal injection of DMSO versus 10 mg/kg or 40 mg/kg BK50118-C for 7 days. WB of midbrain lysates showed that human alpha-synuclein was significantly reduced at the above dosages (FIG. 4A, B 1st blot). Immunoprecipitation of alpha-synuclein protein from midbrain extracts followed by ubiquitin WB (FIG. 4B, 2nd blot) showed increased ubiquitination in 40 mg/kg BK50118-C compared to DMSO and 10 mg/kg BK50118-C. Immunoprecipitation of ubiquitin from midbrain extracts followed by alpha-synuclein WB (FIG. 4B, 3nd blot) showed no monomeric alpha-synuclein but increased ubiquitination of alpha-synuclein in 40 mg/kg BK50118-C compared to DMSO and 10 mg/kg BK50118-C. ELISA measurement of alpha-synuclein in the same extracts (FIG. 4C) confirmed that human alpha-synuclein was reduced by about 30% (10 mg/kg) and 56% (40 mg/kg), respectively (FIG. 4C).

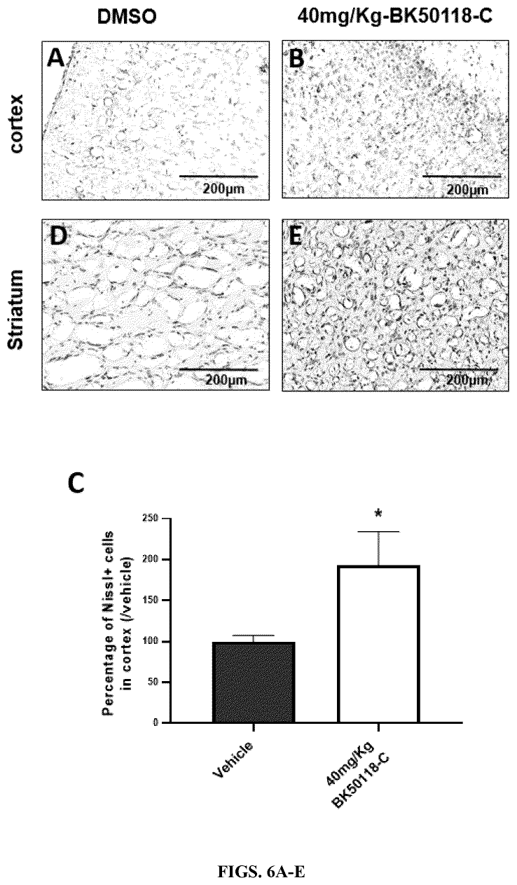

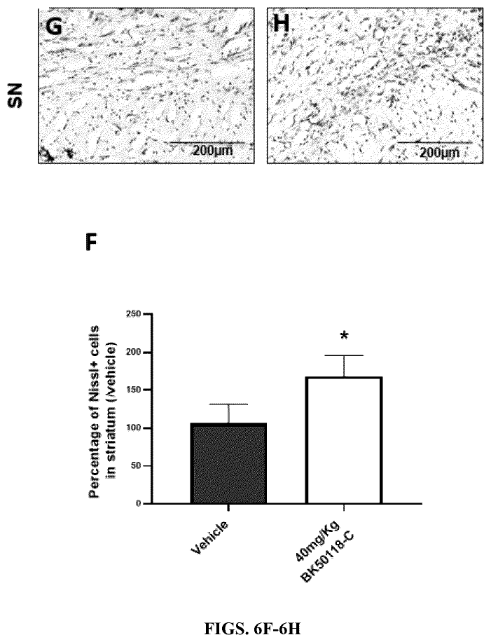

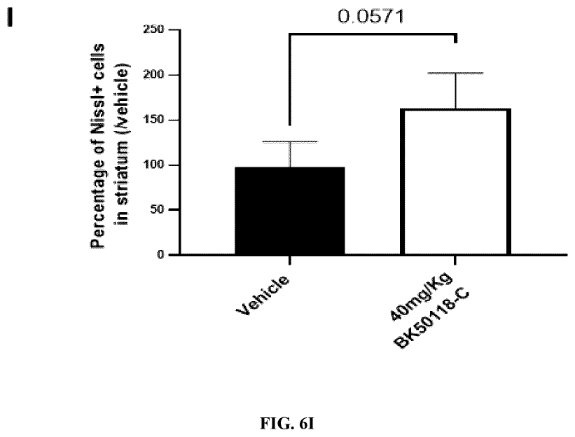

TgA53T mice also demonstrated an elevated state of tauopathy in striata, suggesting that tauopathy is a common feature of synucleinopathies. Therefore, tau levels were also measured. There was no effect on murine tau levels (FIG. 4D). Immunostaining of 20 μm thick brain sections showed human alpha-synuclein staining in both cortex (FIG. 4E, G, I) and striatum (FIG. 4J, L, N) in DMSO treated mice. BK50118-C treatment (40 mg/kg) significantly decreased human alpha-synuclein staining in both the cortex (FIG. 4F, H, I) and striatum (FIG. 4K, M, I) compared to DMSO. Quantification showed that BK50118-C (40 mg/kg) significantly reduced the number of human alpha-synuclein positive neurons by 42% in cortex and 40% in striatum (FIG. 4I, N). Nissl staining showed that BK50118-C significantly increased neuron counts in cortex (FIG. 6B, C), striatum (FIG. 6E, F) and substantia nigra (SN) (FIG. 6H, I) compared to DMSO (FIG. 6A, D, G), as verified by quantification of Nissl+ cells in cortex (FIG. 6C), striatum (FIG. 6F) and SN (FIG. 6I).

### BK50118-C Increases Alpha-Synuclein Ubiquitination and Has Minimal Effects on Tyrosine Hydroxylase Levels in Striatum of TgA53T Mice

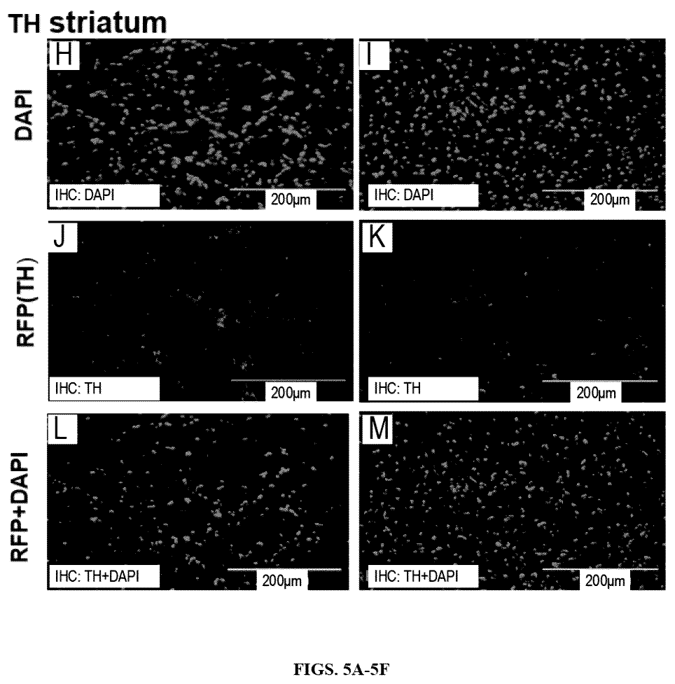

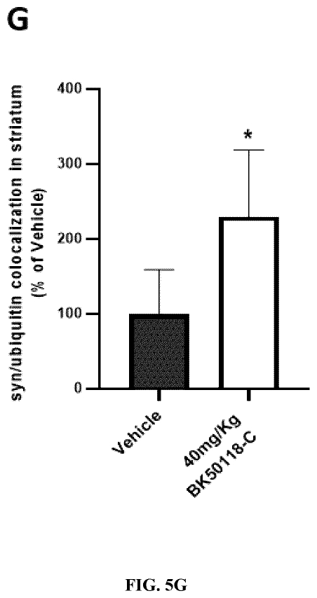

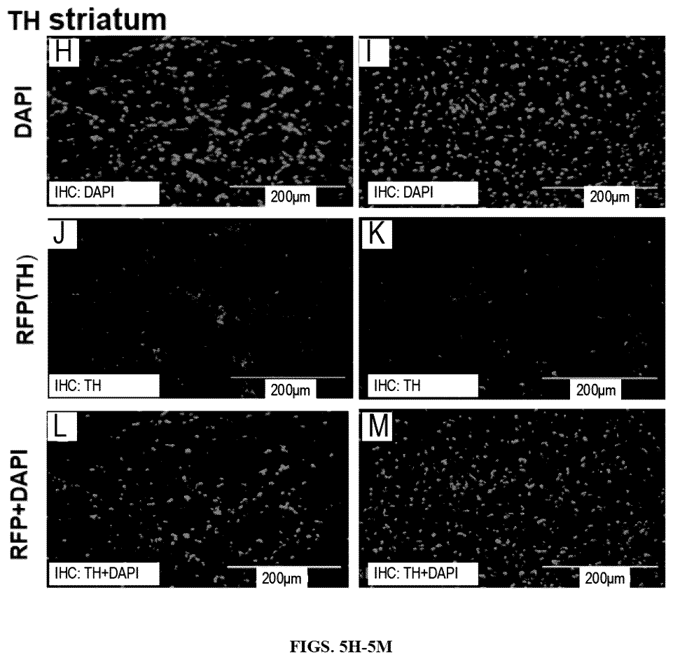

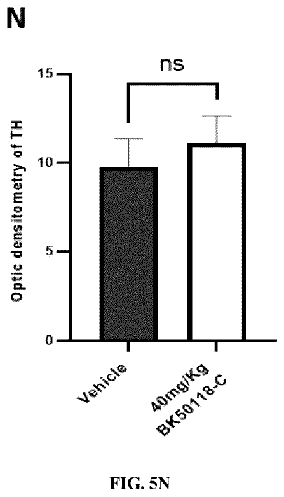

To ascertain the mechanistic effects of BK50118-C on alpha-synuclein clearance, immunostaining was performed in striatum of TgA53T mice. Human alpha-synuclein staining showed the level of human alpha-synuclein in DMSO (FIG. 5A) compared to BK50118-C (40 mg/Kg) treated mice (FIG. 5B). Ubiquitin staining in DMSO treated striatum (FIG. 5C) showed the level of ubiquitin compared to mice treated with 40 mg/Kg of BK50118-C (FIG. 5D). Merged alpha-synuclein and ubiquitin staining in DMSO (FIG. 5E), compared to BK50118-C (FIG. 5F) showed that alpha-synuclein co-localized with ubiquitin in mice treated with BK50118-C. Optic density of alpha-synuclein-ubiquitin staining showed a significant increase (130%) in co-localization in the striatum when mice were injected with BK50118-C compared to DMSO (FIG. 5G). We also performed the staining of tyrosine hydroxylase (TH) in the striatum of TgA53T mice. TH is the enzyme responsible for catalyzing the rate limiting step in synthesis of L-3,4-dihydroxyphenylalanine(L-DOPA) which is a precursor for dopamine (DA). Staining of TH will help to evaluate the status of DA producing neurons in the SN and their terminals in the striatum. Staining of tyrosine hydroxylase (TH) in the striatum of TgA53T mice did not show any noticeable effects in BK50118-C (FIG. 5I, K, M, N) compared to DMSO treated mice (FIG. 5H, J, L, N).

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A compound of the following formula: 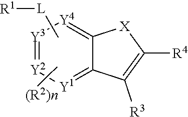
or a pharmaceutically acceptable salt or prodrug thereof, wherein:
n is 0, 1, or 2;
L is S, O, or NR⁷;
X is S, O, NR⁸, or CR⁵═CR⁶;
Y¹, Y², Y³, and Y⁴ are each independently N or CR, wherein R is selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, and wherein at least one of Y¹, Y², Y³, and Y⁴ is N;
R¹ is substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl;
each R² is independently selected from the group consisting of halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl;
R³, R⁴, R⁵, and R⁶ are each independently selected from the group consisting of hydrogen, halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl; and
R⁷ and R⁸ are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

2. The compound of claim 1, wherein the compound has the following formula:

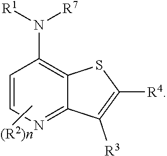

3. The compound of claim 2, wherein n is 0.

4. The compound of claim 2, wherein R³, R⁴, and R⁷ are each hydrogen.

5. The compound of claim 2, wherein R¹ is aryl.

6. The compound of claim 1, wherein the compound has the following formula:

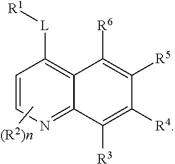

7. The compound of claim 6, wherein n is 0.

8. The compound of claim 6, wherein R³, R⁴, and R⁶ are each hydrogen.

9. The compound of claim 6, wherein R⁵ is halogen.

10. The compound of claim 6, wherein L is NH or O.

11. The compound of claim 6, wherein R¹ is benzyl or phenyl.

12. The compound of claim 1, wherein the compound is selected from the group consisting of:

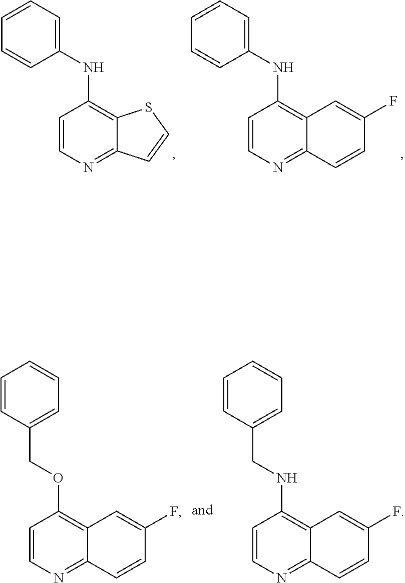 [SMILES: c1ccc(Nc2ccnc3ccsc23)cc1 ; Fc1ccc2nccc(Nc3ccccc3)c2c1 ; Fc1ccc2nccc(OCc3ccccc3)c2c1 ; Fc1ccc2nccc(NCc3ccccc3)c2c1]

13. A compound of the following formula: 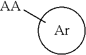
or a pharmaceutically acceptable salt or prodrug thereof, wherein:
AA is an amino acid or ester thereof; and
Ar is substituted or unsubstituted aryl or substituted or unsubstituted heteroaryl,
wherein AA is covalently bonded to Ar through an amino group of AA.

14. The compound of claim 13, wherein the amino acid or ester thereof is a natural amino acid or ester thereof.

15. The compound of claim 13, wherein the amino acid or ester thereof is an unnatural amino acid or ester thereof.

16. The compound of claim 13, wherein the compound has the following formula: 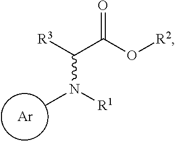
wherein
Ar is substituted or unsubstituted aryl or substituted or unsubstituted heteroaryl; and
R¹ and R² are each independently selected from the group consisting of hydrogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl; and
R³ is hydrogen, halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl.

17. The compound of 16, wherein the compound has the following formula: 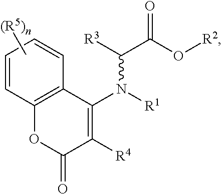
wherein
n is 0, 1, 2, 3, or 4;
R⁴ is selected from the group consisting of hydrogen, halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl; and
each R⁵ is independently selected from the group consisting of halogen, cyano, trifluoromethyl, nitro, hydroxy, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

18. The compound of claim 17, wherein R¹ is hydrogen.

19. The compound of claim 17, wherein R² is methyl.

20. The compound of claim 17, wherein R³ is substituted or unsubstituted benzyl.

21. The compound of claim 17, wherein R⁴ is nitro.

22. The compound of claim 17, wherein n is 0.

23. The compound of claim 13, wherein the compound is selected from the group consisting of:

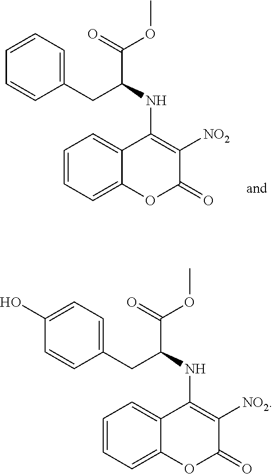 [SMILES: COC(=O)[C@H](Cc1ccccc1)Nc1c([N+](=O)[O-])c(=O)oc2ccccc12 ; COC(=O)[C@H](Cc1ccc(O)cc1)Nc1c([N+](=O)[O-])c(=O)oc2ccccc12]

24. A pharmaceutical composition comprising a compound of claim 1 and a pharmaceutically acceptable carrier.

25. (canceled)

26. A method of treating or preventing a USP13-related disease in a subject, comprising:
administering to the subject an effective amount of a compound of claim 1.

27. (canceled)

28. (canceled)

29. (canceled)

30. The method of claim 26, further comprising administering a second therapeutic agent to the subject.

31. (canceled)

32. (canceled)

33. The method of claim 26, wherein the USP13-related disease is a neurodegenerative disease or cancer.

34. (canceled)

35. The method of claim 33, further comprising administering a second therapeutic agent to the subject.

36. The method of claim 35, wherein the second therapeutic agent is a chemotherapeutic agent or radiation.

37-40. (canceled)
